SEQUENCE LISTING (20 sequences)

SEQ ID NO: 1 - DNA, 22 bp, Artificial Sequence
  A synthetic forward primer for SNP1 developed for the selection of Gn1a
atgcgtgtggcccttgaaaatg

SEQ ID NO: 2 - DNA, 21 bp, Artificial Sequence
  A synthetic reverse primer for SNP1 developed for the selection of Gn1a
agatcttcaaggacgattaag

SEQ ID NO: 3 - DNA, 22 bp, Artificial Sequence
  A synthetic forward primer for SNP2 developed for the selection of Gn1a
attcaagcatgccgtacgtttg

SEQ ID NO: 4 - DNA, 22 bp, Artificial Sequence
  A synthetic reverse primer for SNP2 developed for the selection of Gn1a
agccttcatatgcatgtcgatc

SEQ ID NO: 5 - DNA, 22 bp, Artificial Sequence
  A synthetic forward primer for SNP3 developed for the selection of Gn1a
tccaaaacagtgaaaagcatgc

SEQ ID NO: 6 - DNA, 22 bp, Artificial Sequence
  A synthetic reverse primer for SNP3 developed for the selection of Gn1a
tctagctactacctacactagc

SEQ ID NO: 7 - DNA, 22 bp, Artificial Sequence
  A synthetic forward primer for SNP4 developed for the selection of Gn1a
acttgggcctaatggctagcag

SEQ ID NO: 8 - DNA, 22 bp, Artificial Sequence
  A synthetic reverse primer for SNP4 developed for the selection of Gn1a
tagggtggctatactaaccagt

SEQ ID NO: 9 - DNA, 22 bp, Artificial Sequence
  A synthetic forward primer for SNP5 developed for the selection of Gn1a
agcatgcaaataacgagatgtc

SEQ ID NO: 10 - DNA, 22 bp, Artificial Sequence
  A synthetic feverse primer for SNP5 developed for the selection of Gn1a
ctattttaaattcttgagaggt

SEQ ID NO: 11 - DNA, 23 bp, Artificial Sequence
  A synthetic forward primer for SNP1 developed for the selection of GS3
tggtacacagcatatcatggaac

SEQ ID NO: 12 - DNA, 26 bp, Artificial Sequence
  A synthetic reverse primer for SNP1 developed for the selection of GS3
cagaagtgttataactacatatttgc

SEQ ID NO: 13 - DNA, 22 bp, Artificial Sequence
  A synthetic forward primer for SNP2 developed for the selection of GS3
atctgcaacaaacaagaggatc

SEQ ID NO: 14 - DNA, 26 bp, Artificial Sequence
  A synthetic reverse primer for SNP2 developed for the selection of GS3
cttgagtttccactcacaaacttttc

SEQ ID NO: 15 - DNA, 24 bp, Artificial Sequence
  A synthetic forward primer for SNP3 developed for the selection of GS3
gaaacagctggctggcttactctc

SEQ ID NO: 16 - DNA, 22 bp, Artificial Sequence
  A synthetic reverse primer for SNP3 developed for the selection of GS3
gatccacgcagcctccagatgc

SEQ ID NO: 17 - DNA, 24 bp, Artificial Sequence
  A synthetic forward primer for SNP4 developed for the selection of GS3
ttaggacatatcggcgtgcgttta

SEQ ID NO: 18 - DNA, 24 bp, Artificial Sequence
  A synthetic reverse primer for SNP4 developed for the selection of GS3
cagagaagcatcattgaacgaaca

SEQ ID NO: 19 - DNA, 24 bp, Artificial Sequence
  A synthetic forward primer for SNP5 developed for the selection of GS3
atgcaaccttttctcccttcctat

SEQ ID NO: 20 - DNA, 26 bp, Artificial Sequence
  A synthetic reverse primer for SNP5 developed for the selection of GS3
tctaaaggttaactcagtaaaatcct

(19) United States
(12) Patent Application Publication
(10) Pub. No.: US 2020/0008387 A1
(43) Pub. Date: Jan. 9, 2020

(54) METHOD FOR IMPROVING PLANT VARIETY

(71) Applicant: Institute of Genetics and Developmental Biology, Chinese Academy of Sciences, Beijing (CN)

(72) Inventor: Shaoyang Lin, Beijing (CN)

(21) Appl. No.: 16/038,846

(22) Filed: Jul. 18, 2018

(30) Foreign Application Priority Data
Jul. 9, 2018 (CN) 201810743918.3

(51) Int. Cl. A01H 6/46; C12Q 1/6869; A01H 1/04

(52) U.S. Cl. CPC A01H 6/4636; A01H 1/02; A01H 1/04; C12Q 1/6869

(57) ABSTRACT

The present disclosure relates to a method for improving a plant variety. The present disclosure relates to a method of plant breeding, the method comprising the following steps:
1) Selecting a background variety and a donor variety,
2) Comparing the background variety and the donor variety to identify a module or locus to be improved,
3) Crossing the background variety and the donor variety to obtain a hybrid progeny, backcrossing the hybrid progeny to the background variety to obtain a backcross progeny, and constructing a genetic population using the backcross progeny,
4) Selecting, using molecular markers or a sequencing method, a backcross progeny having chromosomal regions derived from the background variety except for the module or locus to be improved, the molecular markers comprising genome molecular markers and module or locus molecular markers designed according to the selected module or locus,
5) Self-crossing the selected backcross progeny to obtain an improved plant variety.

The present disclosure also relates to a plant variety obtainable by said method.

The method can select a plant in laboratory, obtaining a plant with a definite and improved trait and gene with high breeding efficiency, and achieving division of labour during the breeding process and accumulation of breeding advantages.

### CLAIM FOR PRIORITY

This application claims the benefit of priority of Chinese Application No. 201810743918.3, filed Jul. 9, 2018, which is hereby incorporated by reference in its entirety.

### FIELD OF THE INVENTION

The present disclosure relates to a method for plant breeding. Specifically, the present disclosure relates to a method for improving a plant variety by repairing a parental genomic defect using molecular markers, and relates to an improved plant variety obtainable by the method.

1. Research on Plant Molecular Breeding

Plant breeding relates to a process of selecting specific plants with good traits. This selection involves evaluation of many traits of the breeding population, such as agronomic traits, insect resistance, disease resistance, stress tolerance and quality traits, and the final purpose is to combine the good traits of different varieties into one variety. Traditional breeding methods are based on sexual hybridization breeds through genetic recombination and phenotypic selection. However, the efficiency for selection of complex traits is low, and the methods are susceptible to environmental influences and are time consuming. Since the genetic theories published by Mendel and Morgan, breeders have hoped to switch from phenotypic selection to genotype selection.

1.1 DNA Makers

Since Bernatzky and Tanksley (Bernatzky and Tanksley, 1986) constructed the first RFLP (restriction fragment length polymorphism) marker map of crops, DNA marker research has made great progress. Subsequently, PCR-based DNA labeling technology appears, such as RAPD (random amplified polymorphismic DNA) (Williams et al., 1990), SSR (simple sequence repeats) (Akkaya et al., 1992), AFLP (amplified fragment length polymorphism) (Vos et al., 1995) and so on. RFLP and SSR markers are representative of first- and second-generation molecular markers, respectively, and are widely used to aid in the selection of target traits (Jiang et al., 2012a; Wang et al., 2016). In particular, SSR markers have many advantages such as good reproducibility, co-dominant inheritance, simple operation, low price, and high polymorphism. The disadvantages of SSR markers are that the identification of polymorphic markers between parents is inefficient; the detection of markers requires electrophoresis, which is time consuming and laborious, and developable markers cannot be found in some genomic regions. SNP markers derived from single-base variation are the third-generation DNA markers with the is largest number, the highest abundance, and even distribution throughout the genome. Almost any gene and locus can be tracked with SNP markers and the detection of SNP markers does not require electrophoresis, and has many advantages such as high throughput and rapid detection. It has been adopted by more and more laboratories.

1.2 Application of Molecular Markers in Breeding

Breeders often use good natural variations or artificially created genetic variations to breed new crop varieties that are more in line with human needs. This selection based on crop phenotype is called phenotypic selection. However, the phenotype of a plant depends not only on its genotype, but also on the interaction of the environment or genotype with the environment. Therefore, the phenotype does not respond well to genotypes. Some trait phenotypes are difficult to evaluate (such as the traits of root), time-consuming and laborious (such as physiological and biochemical traits), and evaluation of disease resistance and insect resistance requires specific conditions for testing. Yield traits of materials planting in artificial climate chambers or greenhouse cannot be evaluated. Moreover, it is necessary to see the phenotype after the plant matured for grain traits and the like, which misses the selection of ideal plants for hybridization in the same generation. And the selection based on molecular markers has many advantages: first, it can replace time-consuming and laborious phenotypic evaluation, especially for traits that need to be evaluated in specific growth periods, specific environments, specific locations and seasons; second, it can speed up the background recovery ratio of the recurrent parents and shorten the breeding period; third, the selection can be made in the early generations or in the seedling stage of the crops to reduce the size of the population so as to reduce the burden of later work. Therefore, the use of genotypes to select target traits has brought good news to breeders.

1.3 Foreground Selection

Indirect selection of target traits using molecular markers linked to the target favorable allele/QTL is called molecular markers-assisted selection (MAS). This concept was first proposed by Tanksley in 1983, and Hospital and Charcosset (1997) refer to it as foreground selection. When the target traits are difficult to evaluate (for example, the evaluation of disease resistance and insect resistance requires specific environment and conditions and requires a lot of manpower and material resources), or when multiple favorable traits need to be introgressed at the same time, the foreground selection is more advantageous than the phenotypic selection. The efficiency of foreground selection is related to the genetic distance between the linkage marker and the target gene/QTL. The smaller the genetic distance, the higher the accuracy of selection. When only the linkage marker on one side of the target gene is used for selection, the selection error is often caused by the recombination between the marker and the gene. The use of the linkage marks on both sides at the same time can greatly improve the accuracy of the selection. Genetic markers or functional markers (Andersen and Lubberstedt, 2003) based on differences in the sequence of different alleles within the target gene are more intuitive and accurate in selecting the target gene, because the functional markers are co-segregated with the target gene, selection errors resulting from the recombination between the markers and the genes are avoided. In addition, the functional markers are derived from a polymorphic sequence within the target gene that directly causes a phenotypic difference, and thus whether the target allele is contained on a different genetic background can be determined. The premise of selecting to a gene of interest using a functional marker is that the gene has been cloned and the function of the gene and the polymorphic sequence within the gene that causes phenotypic differences are determined.

2. Research on Rice Breeding

Rice is one of the most important cereal crops worldwide, and more than half of the global population depends on rice as a staple food. The continuous growth of the population and the continuous decline in the area of cultivatable land are making the global food supply and demand situation increasingly tense. In the past more than half a century, the green revolution marked by the application of the semi-dwarf gene sd1 (Sasaki et al., 2002; Spielmeyer et al., 2002) and the promotion of hybrid rice in Southeast Asia have greatly improved food production. However, studies have shown that there is no significant increase in corn, rice, wheat and soybean production in about 24% to 39% of the world's food-growing areas (Ray et al., 2012). The top three rice producers, India, China and Indonesia, experienced yield stagnation in more than 37%, 78% and 81% of their respective rice growing areas from 1961 to 2008 (Ray et al., 2012).

2.1 Although Rice Yield Traits are Complex, they have Recently Advanced Rapidly.

The yield traits of rice are a complex quantitative trait. The yield per plant is mainly composed of three parts: effective tillers per plant, grain number per panicle and grain weight. (Sakamoto and Matsuoka, 2008; Xing and Zhang, 2010). The effective tiller per plant depends on the ability of rice plants to produce tillers, including primary tillers, secondary tillers, and tertiary tillers. The grain number per panicle is composed of spikelet number and the seed setting rate. The spikelet number is further determined by the number of primary branches and secondary branches. (Xing and Zhang, 2010). Grain weight is mainly affected by grain size, and grain size is determined by grain length, grain width, grain thickness and solidity. The yield traits are mutually constrained and interact with each other and are typical quantitative traits controlled by multiple genes. The diversity of genetic composition results in large differences in yield traits that ultimately lead to differences in rice yield. The study of quantitative trait locus (QTL) of rice yield traits provides a good theoretical basis for high-yielding rice breeding.

2.2 QTLs Controlling Rice Yield Traits

QTL mapping is an effective strategy for resolving the genetic basis of yield traits. Although thousands of QTLs have been reported so far (http://www.gramene.org/qtl), since QTL is a statistical concept, its authenticity requires further experimental verification. QTL verification commonly uses the following three methods: the first is to construct QTL-based near isogenic lines (NILs) to eliminate the interference of the genetic background, thereby decomposing the target site into a single Mendelian genetic factor. Most QTLs are currently validated and cloned using this method (Che et al., 2015; Wang et al., 2015a; Zuo and Li, 2014), the disadvantage is that it takes a lot of time and labor to clone a single QTL. The second is to construct a set of continuous chromosome segment substitution lines, each substitution line is made by introducing a small segment of the chromosome from the donor on the genetic background of the recurrent parent, these introgressed small segments can be combined to cover the entire donor genome, which is equivalent to constructing a library of chromosome segments from the donor parent on the genetic background of the recurrent parent. The difference in trait between the introgressed line and the recurrent parent can be considered to be caused by the introgressed chromosomal segments of the donor. Moreover, QTLs with less effect which can not be detected in the primary mapping population such as F₂, BC₁F₁, DH, RIL and so on can be detected with such genetic material. The third is the advanced backcross QTL analysis (AB-QTL) proposed by Tanksley and Nelson (1996), which provides good materials for QTLs cloning and expands genetic resources to improve target traits with more abundant genetic diversity between species (Frary et al., 2000; Li et al., 2005). The currently cloned genes/QTLs, especially the genes/QTLs cloned by the method of map-based cloning, is few, and the genes/QTLs that can be used for breeding is fewer. Here we mainly review some genes/QTLs that have important application value in actual production.

2.3 Grain Number Per Panicle

The rice panicle is composed of cob, primary branch, secondary branch and spikelet. Panicle differentiation is mainly characterized by the formation of branches and spikelets. The grain number per panicle is determined mainly by the length of the panicle, the number of branches, and the density of the grain. Gn1a (grain number1a) is the first cloned major QTL for controlling the grain number per panicle, and alleles from the variety Habataki increased the grain number per panicle. Gn1a was finely mapped to a 6.3-kb interval using 13,000 F₂ plants produced by a single NIL containing Gn1a. This region of the 6.3-kb interval has only one open reading frame (ORF) encoding a OsCKX2 gene highly homologous to cytokinin oxidase/dehydrogenase. Sequence analysis indicated that compared to Koshihikari, the OsCKX2 gene in Habataki lacks 16 and 6 bases in the 5′-UTR and the first exon, respectively, and has 3 base substitutions in the 1st and 4th exons, resulting in amino acid changes. The variation of these DNA sequences leads to a decrease in the expression of OsCKX2, which leads to the accumulation of cytokinins in the lateral meristem, which in turn increases the number of spikelets, increases the grain number per panicle, and ultimately increases the yield per plant of rice (Ashikari et al., 2005). By comparing the promoter regions, 5′-UTR and coding regions of Gn1a in 175 cultivated rice and 21 wild rice, Wang et al. (2015) found that according to the sequence difference of the encoded amino acids, Gn1a has 14 allelic variations in the cultivated rice, which are named AP1-AP14. Among them, the three allelic variations of AP3, AP8 and AP9 occur most frequently in the cultivated rice, and the two allelic variations of AP8 and AP9 are mainly found in indica rice, which is rare in japonica rice. In wild rice, Gn1a also has 9 other allelic variations, named AP15-AP23 (Wang et al., 2015). These different allelic variations provide abundant genetic resources for the improvement of rice grain number.

Ghd7 (grain number, plant height and heading date7) is a multi-effect QTL that controls the grain number per panicle, plant height and heading date. Using the F_{2:3} and RIL populations constructed by Zhenhui 97 and Minghui 63, Ghd7 was located on chromosome 7 of rice and then finely mapped to a 79-kb interval using NIL containing Ghd7 (Xue et al., 2008).). The full-length cDNA of Ghd7 from the donor parent Minghui 63 is 1013-bp, encoding a nucleoprotein consisting of 257 amino acids and including the CCT (CO, CO-like and timing of CAB1) domain which has great similarity with the CCT domain of Arabidopsis CO protein, but is obviously different. The expression and function of Ghd7 are regulated by photoperiod. Under long-day conditions, enhanced expression of Ghd7 can significantly delay the heading date; significantly increase plant height and grain number per panicle, while natural mutants with reduced function can be planted to temperate zone and even region of higher latitudes. Therefore, Ghd7 plays a very important role in increasing the potential and adaptability of global rice production. In addition to regulating the flowering time of rice and affecting the plant height and yield of rice, Weng et al. (2014) found that Ghd7 is involved in the regulation of rice hormone metabolism, biotic stress and abiotic stress, in addition to regulating flowering time. For example, drought, abscisic acid (ABA), jasmonic acid (JA) and high temperature can inhibit the expression of Ghd7, while low temperature promotes the expression of Ghd7. Overexpression of Ghd7 increased the sensitivity of rice to drought, while knockdown of Ghd7 enhanced the of rice to resist drought stress (Weng et al., 2014).

Ghd8/DTH8 is a multi-effect QTL that regulates rice yield, plant height and heading date, the transcription of Ehdl and Hd3a can be down-regulated to delay the flowering of rice under long-day conditions, but promote the flowering of rice under short-day conditions. Ghd8/DTH8 can up-regulate the expression of the gene MOC1 that controls rice tillering and lateral branch development, thereby increasing the number of tillers, primary branches and secondary branches (Wei et al., 2010; Yan et al., 2011).

DEP1 is a major QTL that controls the yield traits of rice and encodes a protein with a similar functional domain to phosphatidylethanolamine binding proteins. The dominant allele at this locus is a gain-of-function mutation. The DEP1 mutation can promote cell division, reduce the length of the neck-panicle node and make the panicle of the rice dense, increases the number of branches, and increases the grain number per panicle, thereby promoting rice yield (Huang et al., 2009). In addition, the DEP1 gene can also regulate nitrogen use efficiency in rice (Sun et al., 2014). The analysis found that the mutant DEP1 gene is widely distributed in the erect and semi-erect panicle-type high-yielding rice varieties widely cultivated in the northeast and the middle and lower reaches of the Yangtze River in China, indicating that the DEP1 gene has played a key role in rice yield increase in China. The study also found that DEP1 gene not only promotes rice yield, but also plays a role in the yield promotion of other crops such as barley and wheat, indicating the important application value of this gene in the molecular breeding of crops for high-yielding (Huang et al., 2009).

NOG1 is located on the long arm of chromosome 1 and encodes the enoyl-CoA hydratase in the fatty acid β-oxidation pathway. A 12-bp transcription factor binding site in the promoter region has a copy number variation, and there is only one 12-bp functional sequence in wild rice and low-yielding rice varieties, while in high-yielding varieties there are two closely linked 12-bp sequences. 12-bp insertion can increase gene expression levels, reduce levels of fatty acids and jasmonic acid in plants, increase the grain number per panicle, increase yield, and not affect plant height, heading date, panicle number, grain weight and other traits (Huo et al., 2017). In addition, Bai et al. (2017) found that OsBZR1 inhibits the expression of FZP by binding two copies of the 18-bp silencer sequence upstream of the start codon of the rice panicle developmental gene FZP, thereby increasing the grain number per panicle, but at the same time also leads to a decrease in thousand grain weight (Bai et al., 2017). The cloning of these genes not only provides an important gene for breeding high-yielding rice varieties, but also provides new clues for revealing the molecular mechanism of regulation of rice yield traits.

2.4 Grain Weight

Rice grain shape has always been an important target trait for breeding improvement. This is mainly because grain size determines grain weight and thus affects rice yield. It is also closely related to the appearance quality and food quality of rice. Grain weight is a complex quantitative trait determined by three factors: grain length, grain width and grain thickness/grain filling. GS3 is the first cloned QTL to control rice grain size and is a major QTL located in the near centromere region of chromosome 3 controlling the rice grain weight and grain length. It also affects the grain width and grain filling of rice. Minghui 63 with large grain was used as a recurrent parent and backcrossed continuously with Chuan 7 with small grain to construct a NIL containing GS3, and genetic analysis of 201 random plants from BC₃F₁ plants with GS3 and heterozygous target segments found that GS3 can explain 83.4% grain weight variation and 95.6% grain length variation (Fan et al., 2006). Using 5740 BC₃F₂ plants from a NIL containing GS3, GS3 was finely mapped to a 7.9-kb interval with a full-length cDNA of 956-bp containing 5 exons encoding a transmembrane protein of 232 amino acids, the protein product comprises the following four domains: N-terminal PEBP domain, a transmembrane region, cysteine-rich homologous region of the tumor necrosis factor receptor/nerve growth factor receptor (TNFR/NGFR) family and a C-terminal von Willebrand factor type C (VWFC module). Sequence analysis indicated that compared with the variety with small grain, the codon TGC of the 55th cysteineencoded by the second exon of GS3 in the variety GS3 with large grain was mutated to the stop codon TGA, resulting in early termination of protein translation and thus deletion of 178 amino acids, which in turn make the PEBP-like domain defective and lack three other functional domains. This suggests that GS3-encoded proteins negatively regulate grain weight (Fan et al., 2006).

The OSR (organ size regulation) domain was formerly known as the PEBP domain, but in recent database software analysis it was found that GS3 does not belong to the PEBP protein family, and the putative PEBP domain of the GS3 was found to be approximately one-third long of the whole PEBP by comparison, with only 20.3% to 28.4% similarity (Mao et al., 2010). At the same time, Mao et al. analyzed the relationship between the function of four domains of GS3 and the size of rice grain. It was found that these domains play different roles in regulating grain size: it is necessary for the OSR domain to function as a negative regulator. The wild-type alleles correspond to the formation of medium-length grain, and loss of the structural function of OSR leads to the formation of long grain; the C-terminal TNFR/NGFR and VWFC domains show inhibition of OSR function, and the loss-of-function mutations of these two functional domain produce very short grain (Mao et al., 2010). The clarification of these mechanisms has important application value for designing rice varieties that meet the needs of different consumer groups.

GW2 is an earlier reported QTL controlling rice grain width and grain weight, encoding a circular E3 ubiquitin ligase localized in the cytoplasm, and constitutively expressed in different tissues of rice. This cyclic E3 ubiquitin ligase negatively regulates cell division by anchoring its substrate to the proteasome for degradation. The transcription of an allele of GW2 was terminated due to deletion of one base on the fourth exon, thus encoding a product that is shortened by 310 amino acids. It is speculated that the loss-of-function of GW2 cannot transfer ubiquitin to the target protein, so that the substrate that should be degraded cannot be specifically recognized, thereby activating the division of the cells of the outer shell or the spikelet, thereby increasing the width of the outer shell of the spikelet. Indirectly, the filling rate is also increased, and the size of the endosperm is also increased. Finally, the grain width, grain weight and yield per plant are increased (Song et al., 2007).

GW5/qSW5 is a QTL that controls rice grain width and grain weight and has a strong effect. It is common in rice resources, has little environmental impact and has a high contribution rate to the traits of grain shape. It has important application value for cultivating high quality and high yield rice varieties. As early as 2008, Wan Jianmin and the Japanese Yano research team successfully located the GW5/qSW5 locus in the same interval on the short arm of chromosome 5. The study found that compared with the varieties with slender grain, the varieties with wide grain had a 1212-bp deletion, which was associated with the trait of wide grain, and verified that the deletion was selected with high intensity during rice domestication and breeding improvement to promote rice production (Shomura et al., 2008; Weng et al., 2008). However, the GW5/qSW5 candidate genes predicted by the two research teams were different, and no functional verification results were reported for the predicted genes. Therefore, the functional genes for the GW5/qSW5 locus need to be further clarified. The latest study clarified that a gene encoding calmodulin at about 5-kb downstream of the 1212-bp deletion region can significantly affect rice grain width and is a candidate gene for the GW5/qSW5 locus, still named GW5 (Liu et Al., 2017). This gene is mainly expressed in the glume of rice grain development. The 1212-bp deletion present in the varieties with wide grain mainly regulates the grain width by affecting the expression level of GW5. Further studies have found that GW5 protein is localized on the plasma membrane and interacts directly with glycogen synthase kinase 2 (GSK2), a key kinase in the brassinosteroids (BR) signaling pathway. Inhibition of phosphorylation by GSK2 of two downstream transcription factors OsBZR1 (Oryza sativa BRASSINAZOLE RESISTANT1) and DLT (DWARF AND LOW-TILLERING) makes non-phosphorylated OsBZR1 and DLT accumulate and enter the nucleus, where they regulate the expression of response genes downstream of BR and then regulate the growth and development process of rice grain width and grain weight. The researchers also found that knocking out the GW5 gene by CRISPR technology can increase the grain width and grain weight of other rice varieties without 1212-bp deletion, and increase yield (Liu et al., 2017). The above results reveal a new mechanism of BR signaling pathway and grain development regulation in rice, and provide new ideas for promoting the yield of other cereal crops.

The above-mentioned cloned genes GS3, GW2, qSW5/GW5 controlling the grain size were negatively correlated with the grain shape, that is, the gene expression level increased and the grain size decreased. GS5 is a cloned QTL located on the short arm of chromosome 5 and positively regulating grain size, GS5 encodes a serine carboxypeptidase, and controls the rice grain width, plumpness and thousand grain weight (Li et al., 2011b). Higher GS5 expression levels may be involved in promoting cell cycle and accelerating cell cycle progression, thereby promoting lateral division of rice glume cells, thereby increasing the width of the glume, which in turn accelerates grain filling and endosperm growth, finally increasing seed size and increase grain weight and yield per plant. Two key SNPs (single nucleotide polymorphisms) in the GS5 promoter region cause differential expression of GS5 in rice panicles, which determines the difference in grain size. GS5 promoters were sequenced and compared from 51 rice lines from different regions of Asia. It was found that GS5 has three different haplotypes in nature, namely GS5 large grain haplotype, GS5 medium grain haplotype and GS5 small grain haplotype, corresponding to three different traits of grain width of different strains: wide, medium width and narrow grain shape. Among them, GS5 small grain haplotype is wild type, while GS5 large grain haplotype is a gain-of-function mutant in rice domestication and breeding process. Therefore, GS5 plays an important role in the process of rice domestication and breeding, and contributes a lot to the genetic diversity of rice seed size (Li et al., 2011b).

The grain thickness is mainly affected by the grain filling degree at the time of grain filling, and only a few related genes have been identified so far. GIF1 is the first cloned gene of grain plumpness, located on chromosome 4, encodes a cell wall invertase and regulates sugar transport unloading and filling during rice grain development. The artificial selection makes the GIF1 of modern cultivated rice have strict tissue expression specificity compared with wild rice, which is beneficial to grain filling and thus promote the yield per plant. However, the gene expression site of wild rice is not specific, which is not conducive to grain filling. Overexpression of GIF1 gene in cultivated rice can significantly increase grain filling degree and thousand grain weight. This is also the first demonstration that a domesticated crop gene can improve the agronomic traits of crops through appropriate gene expression regulation, providing a new option for the molecular design breeding for high-yielding rice (Wang et al., 2008). FLO2 plays a key role in regulating rice grain size and starch quality by affecting the accumulation of storage substances in the endosperm. Overexpression of FLO2 can significantly increase the size of rice grains (She et al., 2010). During the development of caryopsis, GIF2 plays an important regulatory role in rice grain filling and starch synthesis, and it is preserved in the selection of modern rice domestication process (Wei et al., 2017). In addition, studies have shown that GW2 can also increase grain filling rate and increase grain weight and yield per plant (Song et al., 2007). Therefore, GIF1, FLO2, and GIF2 are positive regulators of grain filling, while GW2 is a negative regulator.

In addition to the above-mentioned cloned genes/QTLs controlling yield traits, Some important QTLs are cloned, such as GL3.1 (Qi et al., 2012), OsSPL16/GW8 (Wang et al., 2012), TGW6 (Ishimaru et al., 2013), GW7/GL7 (Wang et al., 2015a; Wang et al., 2015b), GL2/OsGRF4/GS2 (Che et al., 2015; Duan et al., 2015; Hu et al., 2015), these genes and their allelic variations provide favorable genetic resources for the design of rice high-yielding molecular modules and breeding.

The systematic analysis of the regulation mechanism of rice yield traits and quality traits provides theoretical support for the molecular design breeding for rice with high-yielding and high-quality. Through the systematic analysis of the ideal plant type IPA1 and analysis and research on genetic regulation network of starch synthesis-related genes affecting rice cooking quality, Zeng et al. used the high-yielding and insect- and disease-resistant rice variety Teqing as receptor and rice variety nipponbare and 93-11 with good appearance and cooking and eating quality as donors to optimize and combine 28 target genes involved in rice yield, rice appearance and quality, cooking and eating quality and ecological adaptability, and simultaneously aggregate the superior alleles of the target genes into the receptor Teqing to design new rice varieties with high-yield and high-quality (Zeng et al., 2017). This study provides a new way for crops to change from breeding methods depending on traditional experience and based on phenotypic traits to targeted, efficient and accurate molecular design breeding.

3. Problems

Although breeding technology research has made rapid progress, most of the crop varieties currently used in production, especially rice varieties are selected using ancient hybrid selection and breeding techniques. Breeders still need hard work, without any support from science and technology, relying on years of breeding experience and selected and cultivated plants one by one in the field under hot weather. Because the selection in the field requires years of experience, the breeders are generally older, which is even more difficult to breed. In order to cultivate an excellent rice variety, the breeder needs to spend ten or twenty years, or even a lifetime, to cultivate a variety. Secondly, the breeding efficiency is low, the breeding time is long, and the quality of the new varieties cultivated is also low. The breeder found a plant with large panicle in the field, but he did not know if the plant was disease-resistant or susceptible. Even if the breeder found a plant with large panicle and with rice blast resistance, he did not know whether the rice quality of this plant was delicious or not, and whether the large panicle and rice blast resistance of this plant can be inherited or not. Therefore, breeders need to select many candidate plants as well as verify the progeny of these plants. This requires a lot of work to identify these plants. It also takes a lot of time to verify whether their target traits are inherited or not. The workload for identification of these progeny is large and take a long time, and more importantly, spending more than ten years, but the number of candidates in the primary selection was insufficient, or important genes or traits were wrongly selected or missed, then the last selected plant and the cultivated variety will be of low quality, even worse than the original parent.

The third problem is that “one variety is selected by one breeder”. Just like the case that one painting is drawn by one painter, this is a big problem on the technical level. It can be said that this is not called breeding techniques but called skills or art. That is to say, in the breeding process, the most important link (selection) is completed by the breeder alone. It is also possible to select alternate plants and the breeders will judge according to their own experience whether the final comprehensive traits are good or not, and whether they can become good varieties. According to the current breeding technology, it is difficult to divide the breeding process, and a breeder is required to carry out the check.

The fourth problem is that thousands of new varieties are registered every year on the website of the New Variety Protection Office. However, there is no information or data on what traits are improved in these varieties, what genes have been improved, and therefore why these varieties are better than their corresponding original varieties. It is in a state of mixed good and bad varieties, causing confusion for farmers or producers. They have no way to distinguish and choose new varieties suitable for their own production areas. It is often happened that new varieties are worse than old ones. The biggest confusion for farmers is that they cannot apply the experience accumulated over years to new varieties. Everyone knows that farmers has rich experiences and wisdom accumulated over years about when to plant, when to fertilize, when to harvest through long-term repeated planting, and understanding the climate, meteorological conditions, land, and nature. However, these experiences and wisdom are accumulated on the varieties they use. If for some reason, for example, the varieties used have decreased disease resistance and need to be renewed, there will be great confusion for them-they don't know whether the accumulated experiences and wisdom can be used in new varieties, and they need to take several years of experimentation and exploration to get a final answer, which is a big loss.

The fifth problem is that breeding techniques can not be accumulated. The cultivation of an excellent variety not only spends most of the breeder's life, but also accumulates the experience and wisdom of the breeder. But even an excellent variety has to be eliminated due to the decline of traits such as the disease resistance. This time means that the experience and wisdom accumulated by the breeder are destroyed. Not only can the breeder's own experience and technology not be accumulated, but the skills, experience and wisdom between the breeders will not be accumulated.

### SUMMARY OF THE INVENTION

The present disclosure provides a method of plant breeding, the method comprising the following steps:

1) Selecting a background variety and a donor variety,

2) Comparing the background variety and the donor variety to identify a module or locus to be improved,

3) Crossing the background variety and the donor variety to obtain a hybrid progeny, backcrossing the hybrid progeny to the background variety to obtain a backcross progeny, and constructing a genetic population using the backcross progeny,

4) Selecting, using molecular markers or a sequencing method, a backcross progeny having chromosomal regions derived from the background variety except for the module or locus to be improved, the molecular markers comprising genome molecular markers and module or locus molecular markers designed according to the selected module or locus,

5) Self-crossing the selected backcross progeny to obtain an improved plant variety.

As used herein, a plant variety generally refers to a homogeneous population of a species that exhibits one or more common characteristics and has a heritable difference compared to other varieties. In some embodiments, a plant variety can comprise a cultivar, i.e., a collection of cultivated plant population that is characterized by one or more characteristics that are heritable and remain unique in reproduction.

In this context, the locus that needs to be improved refers to a DNA segment within the genome of a background variety, and the DNA segment comprises a gene or QTL locus that control a poor or undesirable trait. A module to be improved refers to a genomic DNA segment containing a locus (loci) that need(s) to be improved. The improved module refers to an allelic genomic DNA segment contains a gene fragment from the donor that controls a desirable phenotype(s), or from a genome or a variety or a line that improves the module of a background variety. The module can affect a specific trait(s) of the plant as a single unit. The module or locus may be introduced into the background variety from the donor variety to improve a certain trait(s) such as grain weight of the background variety. In some embodiments, the size of the sequence of the module can be adjusted to about 50 kb and 5000 kb or longer as desired. It can be adjusted using molecular markers SNP1, SNP2, SNP3, SNP4, and SNP5. In order not to affect the good traits of the background variety, the size of the module may be shortened as much as possible, but reducing the size of the module may require more work. Therefore, if the gene donor and the background variety are close in genetic relationship, a relatively long module can be introgressed. However, if the genetic relationship between the background variety and the gene donor is relatively remote, the module to be introduced is generally required to be short. In some embodiments, the module size can comprise about 50 kb, 100 kb, 150 kb, 200 kb, 250 kb, 300 kb, 350 kb, 400 kb, 450 kb, 500 kb, 550 kb, 600 kb, 650 kb, 700 kb, 750 kb, 800 kb, 850 kb, 900 kb, 950 kb, 1000 kb, 2000 kb, 3000 kb, 4000 kb, 5000 kb or any length between them. In some embodiments, the module size can exceed 5000 kb. In some embodiments, the module can be recombined between the donor variety and the background variety to be introduced into the background variety from the donor variety to improve a certain trait(s) of the background variety. In some embodiments, the background variety comprises only one module derived from the donor variety, while other chromosomes or chromosome segments other than the module retain the original sequence of the background variety. In the description herein, reference to a locus alone may cover a module, or vice versa.

In some embodiments, the background plant can be an elite major cultivate variety. However, even in the case of good varieties, in the process of production, many traits may have defects (bugs), such as poor disease resistance, low yield, insufficient lodging resistance, too late growth period, etc. In some embodiments, the methods herein identify the loci of these bugs by genomic resequencing or by QTL analysis, and identify donor plant varieties that can repair alleles at these loci. Using backcrossing, molecular markers, the genome of the background variety is engineered to achieve a precise improvement of the bug loci. Therefore, the genome of the background variety can be regarded as computer software, and once a bug is found, it can be modified and updated. Therefore, in some embodiments, an elite major cultivate variety can be selected as a background, and a donor variety having a good trait(s) not possessed by the background variety is selected; particularly take a material having a trait urgently needed to be improved in the background variety as a donor. In some embodiments, the donor variety has an improved trait(s) in certain aspects (for example, disease resistance, yield, lodging resistance, growth period, etc.) compared to the background variety. In some embodiments, a donor variety with improved trait(s) can be selected in one or more specific aspects. In some embodiments, a donor variety with an improved trait is selected in one particular aspect, and then another donor variety with an improved trait is selected in another specific aspect, and the breeding module is continuously updated by repeating the method.

In some embodiments, the molecular markers can comprise two types, one type comprising genomic molecular markers, such as SNP markers covering the entire genome, and the other type comprising module or locus molecular markers designed according to a selected module or locus, such as at least 3 SNP markers located at upstream of the module or locus, within the module or locus, and downstream of the module or locus, respectively, such as SNP1, SNP2, and SNP3. In some embodiments, if 3 SNP markers are selected, SNP1 can be upstream of the module or locus, SNP2 can be within the module or locus, and SNP3 can be downstream of the module or locus. In some embodiments, for example, selecting 5 SNP markers, for example, two of them can be designed upstream (SNP1-SNP2), one of them can be designed within a module or locus (SNP3), and two of them can be designed downstream (SNP4-SNP5).

In some embodiments, plants having crossovers between SNP1 and SNP3 as many as possible and having a high background recovery ratio are selected, and then the target plants are obtained by two consecutive selections in its self-crossing progeny.

In some embodiments, step 2) of the breeding methods provided herein comprises genomic sequencing, comparing sequences needed to be improved of module or locus, such as allelic loci, or performing QTL analysis to identify a module or locus needed to be improved. In some embodiments, the methods herein comprise genomic resequencing of the background variety and the donor variety, designing molecular markers using sequencing information, and comparing the alleles. In some embodiments, the methods herein can also comprise QTL analysis of plant varieties, confirmation of the bug loci, and identification of allelic loci that can repair the bug.

In some embodiments, the present disclosure can accurately and controllably improve the variety indoors using the method of genome upgrading, and overcome the problems of large workload, unpredictability, and non-repeatable in the traditional breeding methods. In some embodiments, the method comprises selecting a modified module or locus using molecular markers. In some embodiments, the molecular markers in the breeding methods provided herein comprise RFLP, RAPD, SSR, AFLP and SNP, preferably the molecular marker(s) comprise a SNP marker(s); the module or locus molecular marker(s) comprise at least 3 molecular markers, for example 3, 4, 5, 6, 7, 8, 9, 10, 20, 30, 40, 50, 60, 70, 80, 90, 100 or more molecular markers, upstream of the module or locus, within the module or locus, and downstream of the module or locus. In some embodiments, the methods herein comprise genomic resequencing of the background variety and the donor variety, designing single nucleotide polymorphism (SNP) marker primers between the two, using high resolution dissolution curve analysis (HRM) to screen the designed primers, selecting markers with true polymorphism between the two parents, and then selecting against the whole genome. In some embodiments, the methods herein simultaneously use 3 to 100 or more (3, 4, 5, 6, 7, 8, 9, 10, 20, 30, 40, 50, 60, 70, 80, 90, 100 or more) SNP markers based on sequence polymorphism between the two parents upstream and downstream of the target locus, and said SNP markers are used to select target locus and/or to minimize linkage drag.

In some embodiments, the donor variety provided herein has an improved trait compared to the background variety, the trait being not particularly limited as long as the improvement is advantageous. In some embodiments, the trait comprises for example yield traits (such as high yield, stable yield and traits of efficiency of light use), quality traits (such as amino acid composition, sugar composition, protein composition, lipid composition, trace element composition and harmful components composition such as protease inhibitors, allergen proteins and hydrolases) and stress resistance (such as disease resistance, antibacterial, antiviral, herbicide resistance, drought resistance, high temperature resistance, cold resistance, and nutrient utilization traits).

In some embodiments, the plant is not particularly limited in view of the fact that the method herein is a general breeding method. In some embodiments, the plants can be a crop plant. In some embodiments, the plants may comprise Oryza sativa, Zea mays, Triticum aestivum, Phaseolus vulgaris, Glycine max, Brassica spp., Gossypium hirsutum, Helianthus annuus.

In some embodiments, the plant comprises rice, and the traits can be a trait controlled by a quantitative trait locus (QTL). In some embodiments, the improved module or locus comprises grain number per panicle loci such as Gn1a, Ghd7, Ghd8/DTH8, DEP1, and NOG1, grain weight loci such as GS3, GW2, GW5/qSW5, GS5, GIF1, and FLO2, as well as other loci such as GL3.1, OsSPL16/GW, TGW6, GW7/GL7, and GL2/OsGRF4/GS2.

In some embodiments, step 3) comprises construction of a BC₃F₁ or later population by crossing the background variety and the donor variety and continuous backcrossing for 3 or more generations, detection of the genotype of the BC₃F₁ or later population by whole genome molecular markers and module or locus molecular markers, selection of plants of the BC₃F₁ or progeny population that have introgressed modules and loci from the donor variety and have the highest recovery ratio, and then self cross of the selected BC₃F₁ or later population to obtain a BC₃F₂ or progeny population. In some embodiments, BC₃F₁ plants having a background recovery ratio greater than 85%, such as greater than 90%, such as greater than 92%, are selected. In some embodiments, BC₃F₂ plants having a background recovery ratio greater than 95%, such as greater than 96%, are selected. In some embodiments, BC₃F₃ plants having a background recovery ratio greater than 97%, such as greater than 98%, such as greater than 99%, are selected. In some embodiments, BC₄F₂ plants having a background recovery ratio greater than 98%, such as greater than 99%, such as greater than 99.5%, are selected.

In some embodiments, the step 3) comprises selection of plants with gene crossovers on one side of the target module or locus in the BC₃F₂ or later population by the molecular markers designed according to the selected module or locus, self cross of the plants with gene crossovers to obtain a BC₃F₃ or later population, selection of plants with gene crossovers on the other side of the target module or locus in the BC₃F₃ or later population, elimination of all other non-target chromosome fragments on the genetic background using the principle of backcrossing or self-separation, selection of plants that is introgressed in only chromosome fragments of the target module or locus, self cross of the plants and selection of homozygous fixed plants in the progeny thereof.

In some embodiments, selection started from the BC₃F₁ population by designed molecular markers in the method of the present disclosure. It has been surprisingly found that such selection is very fast and effective. In some embodiments, in order to obtain plants whose improved loci are from a donor variety and the other loci in the genome are of background varieties, the selection is first performed in BC₃F₁ by SNP markers specifically designed for the loci. In some embodiments, for example, molecular markers comprise at least 3 molecular markers, e.g, 5 molecular markers, upstream of the module or locus, within the module or locus, and downstream of the module or locus. In some embodiments, when designing 5 molecular markers, two of them can be designed upstream (SNP1-SNP2), one of them can be designed within a module or locus (SNP3), and two of them can be designed downstream (SNP4-SNP5). In some embodiments, plants who have SNP3 within the locus being the genotype of the locus selected for introduction, have crossovers between SNP1 and SNP5 as many as possible and have a high background recovery ratio is selected, and then the target plants are obtained by two consecutive selections in its self-crossing progeny. In some embodiments, the selection is continued twice. In some embodiments, the first selection comprises in the self-crossing progeny of the plants (BC3F2), selecting plants with crossovers of the target gene (SNP3) with the upstream marker SNP1 and SNP2 or with the downstream marker SNP4 and SNP5; the second selection comprises in the self-crossing progeny of the plants selected in the first step (BC3F3), selecting plants with crossovers between the target gene (SNP3) and the molecular markers at the other end (SNP1, SNP2 or SNP4, SNP5). The plants with crossovers between the target gene (SNP3) and both the upstream (SNP1 or SNP2) and downstream (SNP4 or SNP5) were selected. In some embodiments, the plants having homozygous gene from the donor at the target locus and having the highest recovery ratio are selected as the target plants after self-crossing (BC3F4).

In some embodiments, the method comprises repeating steps 1) to −5) one or more times, each time different modules and loci are selected so as to obtain a plant variety with multiple of improved modules and loci.

In some embodiments, provided herein is a plant variety which is obtainable by the methods described herein and improved compared with a background variety, the improved plant variety comprising improved module or locus compared with the background variety.

The method and plant variety provided herein have one or more of the following advantages:

1) solving the problem of breeder's hard work in the field under hot weather, breeders do not have to select plants in the field, they only need to select plants on the screen of the laboratory or computer.

2) solving the problem of low breeding efficiency, long breeding cycle and low quality of new varieties.

3) solving the problem of “one variety is selected by one breeder” to achieve division of labor

4) solving the problem of farmers and producers confusing for new varieties, and clarifying improved traits and improved genes.

5) solving the problem that breeding technology cannot accumulate and realize the technical accumulation of time and space. The technology of breeders of the same generation, breeders of different generations, breeders of the same research unit, different units, different places, and breeders around the world can be accumulated.

The technical scheme herein is to use the genome of the excellent main plant as the background, and the genome thereof is taken as the computer software, once a bug is found, it is modified and updated.

Like many crops, even in the case of good varieties, in the process of production, many traits will be found with different degrees of bug, such as poor disease resistance, low yield, insufficient lodging resistance, too late growth period, etc. The present disclosure identify the loci of these bugs by genomic resequencing or by QTL analysis, and identify alleles that can repair these loci. Using backcrossing, molecular markers, the genome was engineered to achieve a precise improvement of the bug loci.

In some embodiments, the technical solutions herein may comprise one or more of the following aspects:

1) Selection of background materials: the excellent main plant variety is used as a background to modify the bugs in its genome.

2) Selection of excellent allele donor materials:materials having good traits not possessed by the background variety are selected, particularly take a material having a trait urgently needed to be improved in the background variety as a donor.

3) Genomic sequencing: sequencing of the background variety and the donor materials, designing molecular markers using sequencing information, and comparing the alleles.

4) Taking the background variety as the female parent and the donor material as the male parent for crossing, and backcrossing with the background, using the backcross progeny to construct the genetic population, performing QTL analysis, confirming the bug loci, at the same time identifying and obtaining allelic loci that can repair the bug.

5) Using backcrossing and molecular markers (preferably SNP markers), plants with all the chromosomal regions, except for the vicinity of the bug loci, from the background variety are selected in the backcross progeny.

6) Design 5 markers near the bug region to adjust the size of the donor chromosome segment introgressed, and minimize linkage drag.

7) The plants selected in this way are fixed by self-crossing, and finally an upgraded version of the background can be breeded, which improves the bug of the background variety. An upgraded variety can be breeded for a bug, using such a variety as a module, and it is easy to aggregate two modules together by the hybridization of modules with modules. Similarly, any two or more modules can be aggregated together, and the required modules can be aggregated together. The genome of the background can be changed and designed according to requirements to breed the target variety.

In some embodiments, the methods and plant varieties herein address one or more of the following problems:

1) solving the problem of breeder's hard work in the field under hot weather, breeders do not have to select plants in the field, they only need to select plants on the screen of the laboratory or computer.

The location of the bug can be known by QTL analysis or allelic alignment. This allows plants to be selected by the genotype of the molecular markers distributed throughout the genome. That is to say, the breeder does not have to be in the field, and does not need to select plants, to evaluate the traits of plants or lines in the field under hot weather. Breeders can select plants on the computer screen by analyzing plant genotypes in the laboratory.

2) solving the problem of low breeding efficiency, long breeding cycle and low quality of new varieties.

plants selected according to this method are selected based on the genotype of the chromosome, so there is no environmental error. Therefore, there is no need to worry that the traits of the selected plants are derived from environmental factors and will not be inherited. Therefore, there is no need to select a large number of candidate plants, which can reduce the workload and improve work efficiency. Secondly, because traits except for the traits that need to be improved need not be confirmed and evaluated, the workload is greatly reduced and the work efficiency is improved. Because there is no need to select in the field according to phenotype and selection can be performed at the seedling stage in the laboratory, therefore, rapid generation-adding can be performed in short-day high temperature environment (such as rice), and only a small number of plants are cultivated. Therefore, the breeding efficiency is improved and the breeding cycle is shortened, solving the problem of low breeding efficiency, long breeding cycle and low quality of new varieties

3) solving the problem of “one variety is selected by one breeder” to achieve division of labor.

During the breeding according to this scheme, plants are selected not according to personal experience but the genotypes of the molecular markers, therefore, the breeding work can be distributed to a plurality of people to realize the division of labor. Not only can the work in one module be distributed to multiple people according to their respective proficiency and professional level, which improves the efficiency, and the breeding of multiple modules can be carried out at the same time. Let everyone be responsible for the different processes in multiple module breeding.

4) solving the problem of farmers and producers confusing for new varieties, and clarifying improved traits and improved genes.

Thousands of new varieties are registered every year on the website of the New Variety Protection Office. However, there is no information or data on what traits are improved in these varieties, what genes have been improved, and therefore why these varieties are better than their corresponding original varieties. It is in a state of mixed good and bad varieties, causing confusion for farmers or producers. They have no way to distinguish and choose new varieties suitable for their own production areas. It is often happened that new varieties are worse than old ones. The biggest confusion for farmers is that they cannot apply the experience accumulated over years to new varieties. Everyone knows that farmers has rich experiences and wisdom accumulated over years about when to plant, when to fertilize, when to harvest through long-term repeated planting, and understanding the climate, meteorological conditions, land, and nature. However, these experiences and wisdom are accumulated on the varieties they use. If for some reason, for example, the varieties used have decreased disease resistance and need to be renewed, there will be great confusion for them-they don't know whether the accumulated experiences and wisdom can be used in new varieties, and they need to take several years of experimentation and exploration to get a final answer, which is a big loss. The new varieties cultivated by the method herein not only retain the advantages of the original varieties, but also improve the shortcomings of the original varieties. Therefore, not only the experience and wisdom of farmers or producers can be used in new varieties, but also the problem of the original old varieties can be solved to improve production efficiency.

5) solving the problem that breeding techniques can not be accumulated. The cultivation of an excellent variety not only spends most of the breeder's life, but also accumulates the experience and wisdom of the breeder. But even an excellent variety has to be eliminated due to the decline of traits such as the disease resistance. This time means that the experience and wisdom accumulated by the breeder are destroyed. Not only can the breeder's own experience and technology not be accumulated, but the techniques, experience and wisdom between the breeders will not be accumulated.

The varieties cultivated herein were improved on the basis of the original background varieties. Therefore, the techniques and wisdom of the original breeder can be retained and accumulated. Furthermore, in the process of breeding, the people who participated in, whether they are people of the same generation or people of different generations, whether they are people from the same place or people from different places, the varieties and modules they cultivated can be accumulated, and the modules cultivated by different people can be accumulated in one variety and the method is very simple and very reliable.

### BRIEF DESCRIPTION OF THE DRAWINGS

FIG. 1: Cultivation of modules derived from a single gene donor and aggregation of the modules.

FIG. 2: Module aggregation and chromosome design.

FIG. 3: the population construction for the improvement of grain number per panicle and the selection of single point substitution lines, where KY131=Kongyu 131.

FIG. 4: Gn1a structure and allelic variation of Kongyu 131, GKBR, Koshihikari and Habataki.

FIG. 5: Frequency distribution of background recovery ratios for the BC₃F₁ population. In the figure, the abscissa indicates the background recovery ratio, and the ordinate indicates the number of plants.

FIG. 6: Graphical genotype (GGT) of BC₃F₁-22F01. In the figure, the gray bar represents the chromosome fragment derived from Kongyu 131, the black bar represents the chromosome fragment derived from the donor GKBR, and the white column (labeled as U in the figure) represents that the chromosome fragment type is unknown (the genotype is not determined), the thin black horizontal line represents the position of the SNP marks, and the thick black horizontal line represents the centromere.

FIG. 7: Panicle type of Kongyu 131 and BC₃F₂-LQ96 population. The BC₃F₂-LQ96 population was derived from BC₃F₁-22F01 and consisted of 96 randomly selected self-crossing progeny plants, and was planted in Jiamusi in 2015 with Kongyu 131. The scale bar in the figure represents 5 cm.

FIG. 8: Frequency distribution of five panicle traits in the BC₃F₂-LQ96 population. The black and white arrows in the figure indicate the mean (M1 and M2) of Kongyu 131 and the population, respectively. The phenotypic value of Kongyu 131 is from the mean of 10 Kongyu 131, and the phenotype of BC₃F₂-LQ96 population is the mean of 96 plants within the population.

FIG. 9: Genetic analysis of the genotype and phenotype of the BC₃F₂-LQ96 population. (a-d) LOD values for four panicle related traits. The abscissa indicates 160 SNP markers on 12 pairs of chromosomes. (e-h) Comparison of phenotypic values of four panicle related traits of three genotypes at the Gn1a locus in the BC₃F₂-LQ96 population. KY131 represents Kongyu 131, −/− represents homozygous Kongyu 131 at Gn1a locus, +/− represents heterozygous type at Gn1a locus, and +/+ represents homozygous GKBR type at Gn1a locus. A, B, C, and a, b, and c above the bar graph represents significant differences at the levels of p≤0.01 and p≤0.05, respectively, by the T test (FIG. 9 to continued).

FIG. 10: Graphical genotype (GGT) of selected plants. (a) BC₃F₂-2B09; (b) BC₃F₃-652E09; (c) BC₃F₃-624A05; (d) BC₄F₂-350A09. In the figure, the gray bar represents the chromosome fragment derived from KongYu 131, the black bar represents the chromosome fragment derived from the donor GKBR, and the white column (labeled as U in the figure) represents that the chromosome fragment type is unknown (the genotype is not determined), the thin black horizontal line represents the position of the SNP marks, and the thick black horizontal line represents the centromere.

FIG. 11: Field display of Kongyu 131 and its improved line BC₃F₃-624A05 at the filling stage (Jiamusi, 2016). The left side of the red line is the Kongyu 131, and the right side is the improved line BC₃F₃-624A05.

FIG. 12: Field display of the Kongyu 131 and its improved line BC₃F₃-624A05 at maturity (Jiamusi, 2016). The left side of the red line is the Kongyu 131, and the right side is the improved line BC₃F₃-624A05.

FIG. 13: Plant type, panicle type and grain type of Kongyu 131 and its improved lines BC₃F₃-624A05 and BC₄F₂-350A09. (a) Plant type, Kongyu 131 (left), BC₃F₃-624A05 (right), scale bar 20 cm. (b) Plant type, Kongyu 131 (left), BC₄F₂-350A09 (right), scale bar 20 cm. (c) Panicle type, Kongyu 131 (left), BC₃F₃-624A05 (right), scale bar 5 cm. (d) Panicle type, Kongyu 131 (left), BC₄F₂-350A09 (right), scale bar 5 cm. (e) Grain type, Kongyu 131 (top), BC₃F₃-624A05 (bottom), scale 1 cm. (f) Grain type, Kongyu 131 (top), BC₄F₂-350A09 (bottom), scale bar 1 cm. (a), (c), (e) 2016 Changchun; (b), (d), (f) 2017 Jiamusi.

FIG. 14: Comparison of grain quality traits between Kongyu 131 and its improved lines. (a) Grain appearance of milled rice, 2017 Jiamusi, Kongyu 131 is on the left, 2017 Jiamusi, improved line BC₄F₂-350A09 is on the right, scale bar is 1 cm; (b) polished rice length (n=30); (c) milled rice width (n=30); (d) aspect ratio (n=30); (e) chalky kernel rate (n=500); (f) amylose content (n=4); (g) alkali spreading value (n=14); In b-g, the black column represents Kongyu 131, and the gray column represents the improved line, in which the improved line BC₃F₃-624A05 for 2016 Changchun and 2017 Jiamusi, and the improved line BC₄F₂-350A09 for 2017 Jiamusi.

FIG. 15: GS3 locus sequence comparison between kongyu 131 and BR and SNP Markers used to selecting for gs3 gene from donor, kongyu 131 and BR have a base difference at the second exon as same as difference between Chuan? and Minghui63 from which the GS3 gene was first cloned.

FIG. 16: Graphical Genotype (GGT) of selected individuals or lines. a BC3F1-1. b BC3F2-2. c BC3F3-3. d BC3F4-4. The gray type means the chromosome of kongyu131, and the black represents the fragments from BR.

FIG. 17: QTL analysis indicates that GS3 allelic from donor BR is positively increase grain length in recurrent parent of kongyu 131 background. a QTL analysis with F2 populations. b QTL analysis with BC3F2 populations. c The morphological feature of plant with different genotype at GS3 locus. d The grain length is significantly increased with the donor BR allelic at GS3 locus.

FIG. 18: Grain length and 100-grains weight significantly increased in improved line at GS3 locus compared with kongyu131. a The morphological feature of grain size and plant with different genotype at GS3 locus. b˜e Comparison of grain related traits. grain length and 100-grains weight significantly increased, while grain width and total grain weight per plant increased non-significant. f-j Comparison of main panicle related traits. Primary branch numbers of main panicle increased significantly while panicle numbers per plant decreased greatly and others varied little. k Plant height varied little.

FIG. 19: Field plot trial demonstrates that the primary improved line with GS3 allelic of donor BR at the background of kongyu131 significantly increased grain length and yield compared with the recurrent parent of kongyu131. a Field picture of kongyu131 and the primary improved line. b˜c Grain length and total grain weight per plant increased significantly of the primary improved line compare with kongyu 131. It strongly indicates that the improved line is better than the parent at grain length and yield by using the new method of update design breeding through update the grain length locus GS3.

### DETAILED DESCRIPTION OF ILLUSTRATIVE EMBODIMENT(S)

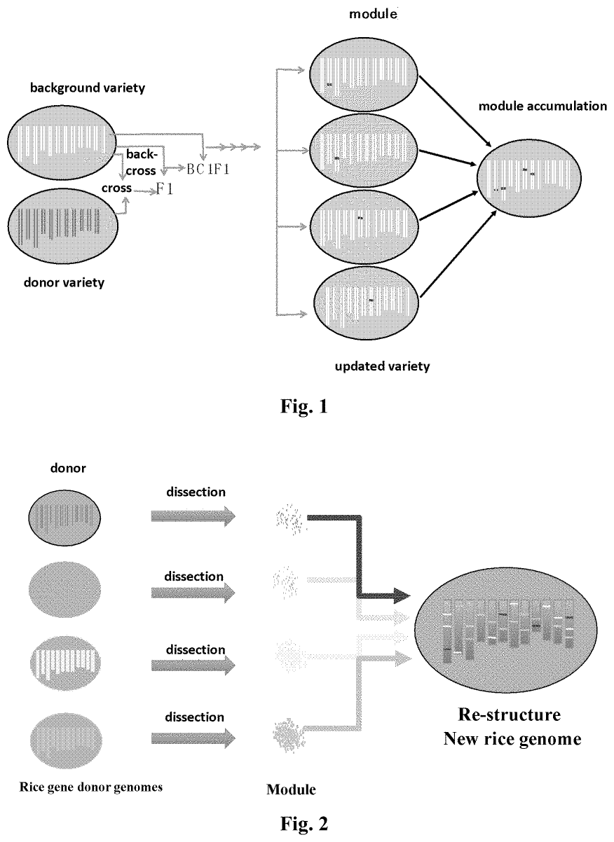

FIG. 1 shows cultivation of modules derived from a single gene donor and aggregation of the modules.

The main plant variety is used as a background variety, which is modified according to the genome defect of the background variety, and the improved line (upgraded variety) (module) of the specific trait of the background variety is cultivated. These modules are aggregated as needed.

FIG. 2 shows module aggregation and genomic design.

After improving the traits of the background and cultivating enough modules, the genome design can be realized. Not only can a single donor be used to cultivate multiple modules, but multiple donors can also be utilized to cultivate more modules.

### EXAMPLE 1

Using high-yielding gene modules to improve and upgrade the main rice variety, Kongyu 131

Methods and Results:

1.1 Experimental Materials

1.1.1 Rice Material

The recurrent parent Kongyu 131 (background variety): it is an early maturity japonica rice variety grown in the high latitude zone, has strong tillering ability, is fertilizer tolerant, lodging resistant and cold tolerant, and requires an active accumulated temperature of 2320° C. Seeding blast grade 9, leaf blast grade 7, panicle neck blast grade 9 are artificially inoculated, and blast grade 9, leaf blast grade 7, panicle neck blast grade 7 are infected naturally. The head milled rice rate is 73.3%, the amylose content is 17.2%, the protein content is 7.41%, and the average yield in Heilongjiang Province is 7684.5 Kg/ha.

Donor parent GKBR: it is an indica rice with large panicles and is blast resistant. The growth period of GKBR in Guangzhou, China, is 113 days in late season, but normal heading does not occur in Heilongjiang Province due to unsuitable photoperiod and temperature conditions.

1.2 Experimental Methods

1.2.1 Parental Resequencing, Sequence Comparison Gn1a and Pi21 Allele and SNP Marker Design

The genomes of rice parental Kongyu 131 and GKBR were re-sequenced by HiSeq 2000 sequencer, and SNP sites between parents were obtained according to the resequencing information. Based on these SNP information, molecular markers were designed for identification of population genotype and plant selection. The sequence of Gn1a of Koshihikari and Habataki (Ashikari et al., 2005) was also downloaded from Genbank (www.ncbi.nlm.nih.gov/genbank) for allele sequence comparison.

The Gn1a sequences of Kongyu 131, GKBR, Habataki and Koshihikari were compared using DNAMAN. SNP markers were designed based on the sequence differences between Kongyu 131 and GKBR. A specific sequence of 22-24 bases was selected as a positive and negative primer on both sides of the SNP. The size of the amplified to fragment was 50-100 bp. Five SNP markers were designed within Gn1a (Table 1). SNP3 was located in Gn1a, SNP1 and SNP2 were located upstream of Gn1a, while SNP4 and SNP5 were located downstream of Gn1a. SNP2 and SNP4 were close to the 5′-UTR and the 3′-UTR of Gn1a. The distance of SNP2 and SNP4 was 5761 bp, while the distance between SNP1 and SNP5 was 856 Kb.

TABLE 1
Sequences of the SNP markers developed for the selection of Gnla

| Markers | Chr. | Position | Forward primer | Reverse primer |
|---|---|---|---|---|
| SNP1 | 1 | 5,006,541 | ATGCGTGTGGCCCTTGAAAATG | AGATCTTCAAGGACGATTAAG |
| SNP2 | 1 | 5,269,396 | ATTCAAGCATGCCGTACGTTTG | AGCCTTCATATGCATGTCGATC |
| SNP3 | 1 | 5,272,491 | TCCAAAACAGTGAAAAGCATGC | TCTAGCTACTACCTACACTAGC |
| SNP4 | 1 | 5,275,157 | ACTTGGGCCTAATGGCTAGCAG | TAGGGTGGCTATACTAACCAGT |
| SNP5 | 1 | 5,862,787 | AGCATGCAAATAACGAGATGTC | CTATTTTAAATTCTTGAGAGGT |

Position refers to the physical location of IRGSP-1.0

1.2.2 Population Construction and Plant Selection for Improved Grain Number Per Panicle

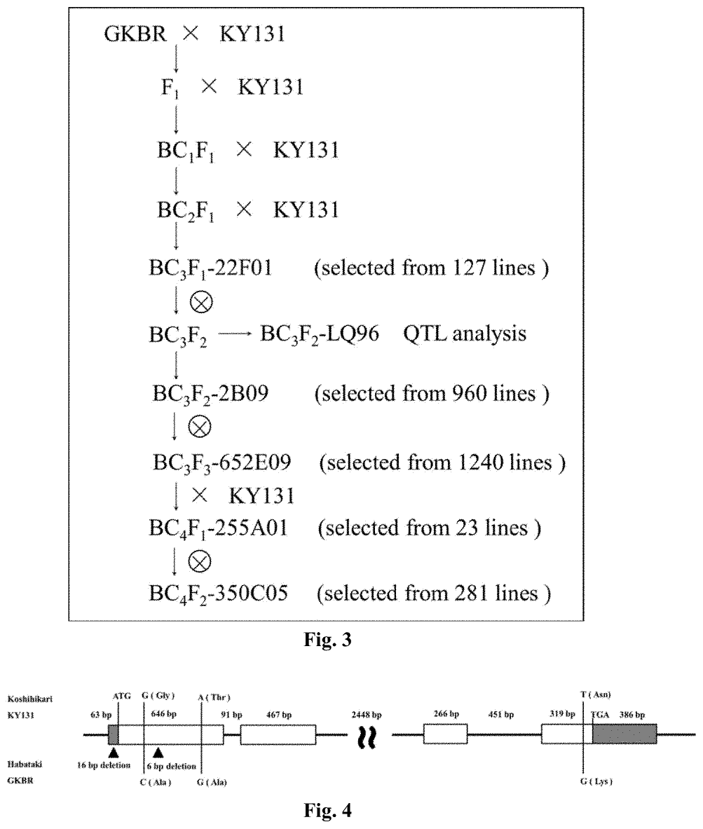

Indica rice GKBR with large panicles was used as a donor and crossed with Kongyu 131 and then backcrossed for 3 generations to produce a BC₃F₁ population that comprised 127 lines. 160 SNP markers designed based on sequence differences between parents were used to detect the genotypes of BC₃F₁ population from which an plant BC₃F₁-22F01 with Gn1a chromosomal fragment of the donor introgressed and the highest background recovery ratio were selected based on the genotype information of SNP1˜SNP5 of Gn1a, and the plant BC₃F₁-22F01 was self-crossed to obtain a BC₃F₂ population. A total of 96 plants were randomly selected from the BC₃F₂ population to form a subpopulation BC₃F₂-LQ96 for QTL analysis validation (FIG. 3). FIG. 3 shows the population construction for the improvement of grain number per panicle and the selection of single point substitution lines, where KY131=KongYu 131.

At the same time, plants with crossovers on one side of the target gene were selected from the large population of BC₃F₂ produced by BC₃F₁-22F01 selfing; then a second crossover and selection was made in the large population of BC₃F₃ produced by self-crossing of the above obtained plants with crossovers, and plants with crossovers on the other side of the target gene were selected; the principle of backcrossing or self-separation was used to exclude all other non-target chromosome fragments on the genetic background, and only plants with small fragments of the target gene introgressed were selected; and finally, homozygous plants were selected in their selfing progeny.

1.2.4 DNA Extraction, PCR and HRM Detection

Genomic DNA from rice leaves was extracted using the quick and easy extraction method described Wang H N, (2013) (Wang H N, Chu Z Z, Ma X L, Li R Q, Liu Y G (2013) A high through-put protocol of plant genomic DNA preparation for PCR. Acta Agron Sin 39:1200-1205). The 10 μl PCR reaction system is as follows: about 50 ng of template DNA, 1 μl of 10× Easy Taq buffer (Transgen Biotech, Beijing, China), 0.2 μl of 2.5 mM dNTPs (Transgen Biotech, Beijing, China), 0.5 U Easy Taq DNA Polymerase (Transgen Biotech, Beijing, China), 0.125 μl 20× EvaGreen (Biotium Inc.). PCR amplification was performed on a 96-well PCR plate, and the reaction system was covered with 10 μl of mineral oil (Amresco Inc.), amplification was performed by a two-step method, 95° C., 5 mins, 1 cycle; 95° C., 15 s, 55˜65° C. (depending on the Tin value of the primer), 30 s, 40 cycles (the PCR has no extension step because the amplicon is small). After the end of PCR, LightCycler® 96 (Roche, Inc.) was used for high resolution melting (HRM) analysis. The SNP genotyping method was the same as (Hofinger et al., 2009)(Hofinger B J, Jing H C, Hammond-Kosack K E, Kanyuka K (2009) High-resolution melting analysis of cDNA-derived PCR amplicons for rapid and cost-effective identification of novel alleles in barley. Theor Appl Genet 119:851-865).

1.2.5 Field Planting of Separated Populations and Investigation of Panicle Traits

The Kongyu 131 was crossed with GKBR and then backcrossed continually to construct a BC₃F₁ population from which an plant BC₃F₂-22F01 with chromosomal fragment of the target gene Gn1a introduced and the highest background recovery ratio were selected, and the plant BC₃F₂-22F01 was self-crossed to obtain a BC₃F₂-LQ96 population which was planted in the rice field in Jiamusi fo Heilongjiang Province (E130° 57′, N46° 23′) in April 2015. Sowing and seedling were carried out on 96-well seeding plates with holes (2.5 mm in diameter) at the bottom. One seed is sown per well, and the embryo is facing upwards. When the seedlings grow to 3-4 leaves, they are transplanted to the rice field, and one plant is inserted into each hole. Each plot contains 8 rows of 12 plants per row, with a plant spacing of 20 cm and a row spacing of 30 cm. The field management is consistent with the local rice field. After maturity, the panicle length (PL), the grain number per panicle (GNP) and the number of primary branches per panicle (NPB) were investigated and the grain density per panicle (GDP) and the density of primary branches (DPB) were calculated as follows: GDP=GNP/PL; DPB=NPB/PL.

1.2.8 Evaluation of Agronomic Traits of Improved Lines BC₃F₃-624A05, BC₄F₂-350A09 and Kongyu 131

On April 18th and Apr. 10, 2016, in Changchun City, Jilin Province (E125° 18′, N44° 43′) and Jiamusi City (E130° 57′, N46° 23′) in Heilongjiang Province, respectivelly, improved lines BC₃F₃-624A05 from Kongyu 131 containing a small Gn1a chromosomal fragment which can be inherited stablely and Kongyu 131 were used for field trials; on Apr. 10, 2017 in Jiamusi City, Heilongjiang Province (E130° 57′, N46° 23′), single point substitution line BC₄F₂-350A09 containing a small Gn1a chromosomal fragment which can be inherited stablely and Kongyu 131 were used for field trials. The field trial used a completely randomized block design with 3 replicates. Each replicates (plot) contains 8 rows of 12 plants per row, with a plant spacing of 20 cm and a row spacing of 30 cm. The field management is consistent with the local regular rice field.

In the trait investigation, 10 plants with normal growth were randomly selected in the middle of each plot; every selected plant had to meet the condition that its surrounding 8 plants exhibited normal growth vigor. Plant height (PH), effective tillers per plant (PNP), Panicle length (PL), number of primary branches per panicle (NPB), grain number per panicle (GNP), grain length (GL), grain width (GW), grain thickness (GT), thousand grain weight (TGW), grain weight per plant, grain water content, and yield per plant (YP) when water content is 15%, density of primary branches (DPB), grain density per panicle (GDP), seed setting percentage (SSP), grain length to width ratio (LWR), and actual yield per plot (AYP) were investigated. The method for trait investigation is shown in Table 2.

TABLE 2
The method for investigation of agronomic traits of
parents, improved lines and BC₃F₂ plants

| Traits | Evaluation method |
|---|---|
| Heading period (DTH, day) | The number of days from soaking to 50% heading of the |
| | plants in the plot |
| Plant height (PH, cm) | From the base of the stem to the neck of the highest |
| | panicle |
| Effective tillers per plant | All the number of tillers with grains |
| (PNP) | |
| Panicle length (PL, cm) | The length from neck of the highest panicle to the grain |
| | on the top (excluding the awn) |
| Number of primary branches | Number of all primary branches of the highest panicle |
| per panicle (NPB) | |
| Density of primary branches | The number of primary branches per panicle divided by |
| (DPB) | the panicle length, that is, the number of primary |
| | branches per panicle per centimeter |
| Grain number per panicle | The number of all grains on the highest panicle, including |
| (GNP) | full grain and empty grain |
| Grain density per panicle | The grain number per panicle divided by the panicle |
| (GDP,/cm) | length, that is, the grain number per panicle per |
| | centimeter |
| Seed setting percentage | The number of full grains divided by the total grain |
| (SSP) | number per panicle multiplied by 100% |
| Yield per plant (YP, g) | Harvest all the grains of a single plant and calculate the |
| | grain weight when the water content is 15%. |
| Thousand grain weight | After taking 500 full grains, weighed and converted to |
| (TGW, g) | thousand grain weight |
| Grain length (GL, mm) | Calculate the mean after measuring the length of 10 full |
| | grains |
| Grain width (GW, mm) | Calculate the mean after measuring the width of 10 full |
| | grains |
| Grain thickness (GT, mm) | Calculate the mean value after measuring the thickness of |
| | 10 full grains with a vernier caliper |
| Actual yield per plot (AYP | After the plants were harvest, the grain weight of the plot |
| kg) | plant was measured and calculated when the water |
| | content was 15%. |

2.1 Improvement of the Gn1a Gene Locus of the Grain Number Per Panicle of Kongyu 131

2.1.1 Sequence Comparison of Gn1a Alleles

Comparison of the Gn1a sequences of Kongyu 131, GKBR, Habataki and Koshihikari showed that the CDS region, 5′-UTR and 3′-UTR sequences of Gn1a of Kongyu 131 and Koshihikari are identical, while the CDS region, 5′-UTR and 3′-UTR sequences of Gn1a of GKBR and Habataki are identical, that is, the Gn1a sequence of GKBR has a 16-bp and 6-bp deletion in the 5′-UTR and the first exon, respectively, relative to Kongyu 131 and the change of three bases in the first exon and the fourth exon resulted in amino acid variation (FIG. 4). The sequence analysis of Gn1a promoter region, 5′-UTR and CDS region of 175 cultivated rice and 21 wild rice according to Wang et al. (2015), the Gn1a allelic variation type of GKBR belongs to AP8, i.e., an allelic variation type with the highest frequency after artificial and natural selection in cultivated rice, and is mainly present in indica rice (Wang et al., 2015) (Wang S, Li S, Liu Q, Wu K, Zhang J, Wang S, Wang Y, Chen X, Zhang Y, Gao C, Wang F, Huang H, Fu X (2015) The OsSPL16-GW7 regulatory module determines grain shape and simultaneously improves rice yield and grain quality. Nat Genet 47:949-954). FIG. 4 shows Gn1a structure and allelic variation of Kong Yu 131, GKBR, Koshihikari and Habataki.

The black vertical line represents that the three base variations of the coding region result in a change in the encoded amino acid, two black triangles represent 16-bp and 6-bp base deletions, and gray rectangles represent 5′-UTR and 3′-UTR, white rectangle represents the exon and the horizontal black line represents the intron.

2.1.2 Detection of Genotype and Background Recovery Ratio

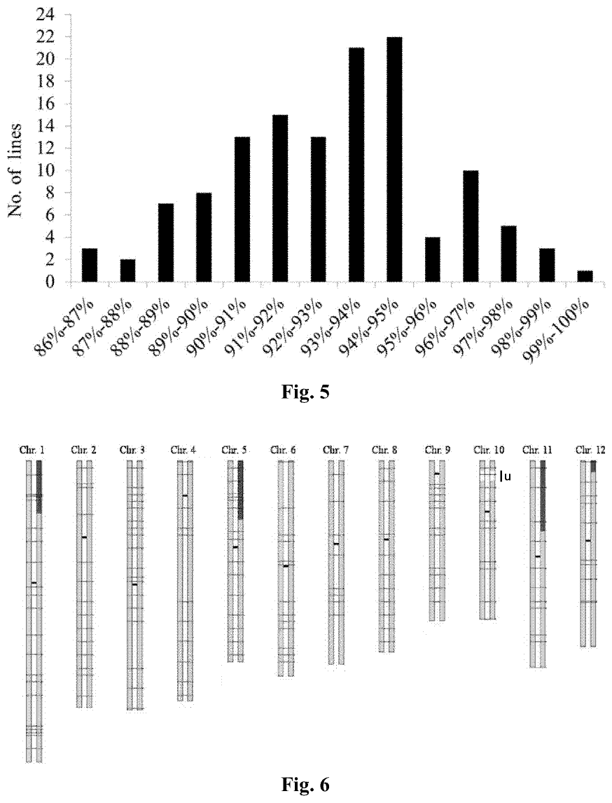

160 SNP markers evenly distributed over 12 chromosomes were used to detect the BC₃F₁ population consisting of 127 lines and the background recovery ratio ranged from 86.3% to 99.5% (FIG. 5) with an average of 92.83%. This value is close to the theoretical background recovery ratio of 93.75% in the BC₃F₁ generation, and the difference could be attributed to unidentified genotypes of some loci in part plants. The genotype of the parent BC₃F₁-22F01 in BC₃F₂-LQ96 population is shown in FIG. 6, and BC₃F₁-22F01 carried one introgressed fragment on chromosome 1, 5, 11 and 12 respectivelly. According to the 160 SNP markers, the average distance between the markers was 2.4-Mb and the background recovery ratio was 96.27%.

FIG. 5 shows the frequency distribution of the background recovery ratio of the BC₃F₁ population, in which the abscissa indicates the background recovery ratio and the ordinate indicates the number of plants.

FIG. 6 shows graphical genotype (GGT) of BC₃F₁-22F01.

In the figure, the gray bar represents the chromosome fragment derived from KongYu 131, the black bar represents the chromosome fragment derived from the donor GKBR, and the white column represents that the chromosome fragment type is unknown (the genotype is not determined), the thin black horizontal line represents the position of the SNP marks, and the thick black horizontal line represents the centromere.

2.1.3 Description and Statistics of Panicle Phenotypes in the Population of Kongyu 131 and BC₃F₂-LQ96

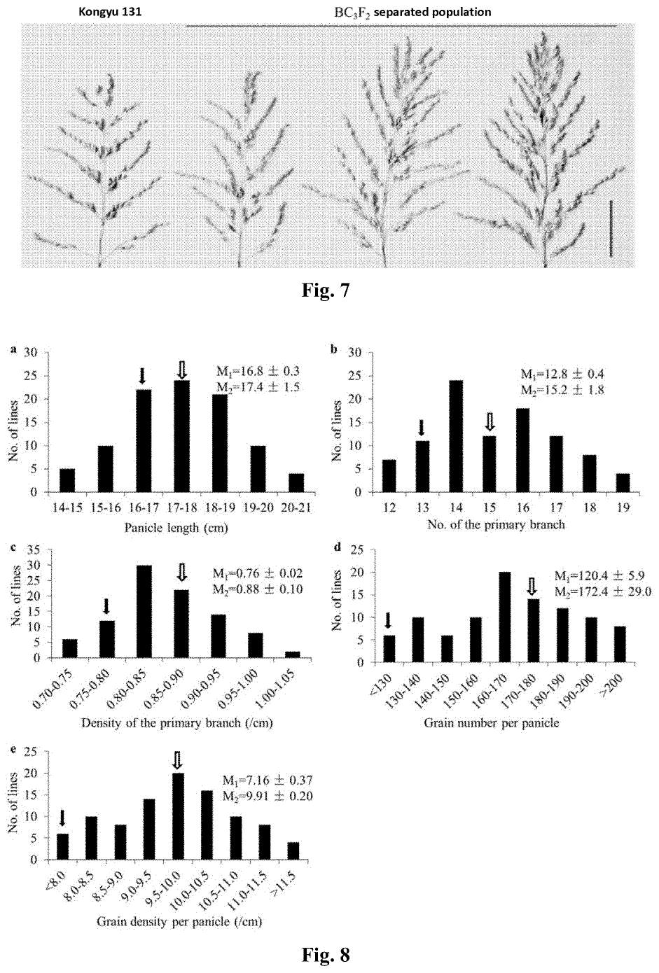

A field phenotypic investigation in the Jiamusi rice field in 2015 revealed a significant separation of the panicle size in the BC₃F₂-LQ96 population (FIG. 7). The frequency distribution of paniclelength (PL), number of primary branches per panicle (NPB), density of primary branches (DPB), grain number per panicle (GNP), and grain density per panicle (GDP) of the BC₃F₂-LQ96 population is shown in FIG. 8. The average value of panicle length, number of primary branches per panicle, density of primary branches, grain number per panicle, and grain density per panicle of the BC₃F₂-LQ96 population increased compared with that of Kongyu 131. The minimum value was close to that of Kongyu 131, and the maximum value increased compared with that of Kongyu 131. It was shown that the chromosome fragment from the donor GKBR can increase the panicle phenotype value.

FIG. 7 shows panicle type of KongYu 131 and BC₃F₂-LQ96 population.

The BC₃F₂-LQ96 population was derived from BC₃F₁-22F01 and consisted of 96 randomly selected selfing progeny plants, and was planted in Jiamusi in 2015 with Kongyu 131. The scale bar in the figure represents 5 cm.

TABLE 3
Phenotypes of Kongyu 131 and random population
BC₃F₂-LQ96 derived from BC₃F₁-22F01

| | KY131 | BC₃F₂-LQ96 population | |
|---|---|---|---|
| Traits | Mean ± SD | Mean ± SD | Range |
| PL (cm) | 16.8 ± 0.3 | 17.4 ± 1.5 | 14.7~20.1 |
| NPB | 12.8 ± 0.4 | 15.2 ± 1.8 | 12.0~19.0 |
| DPB (/cm) | 0.76 ± 0.02 | 0.88 ± 0.10 | 0.73~1.22 |
| GNP | 120.4 ± 5.9 | 172.4 ± 29.0 | 113.0~235.0 |
| GDP (/cm) | 7.16 ± 0.37 | 9.91 ± 0.20 | 7.09~13.81 |

The correlation coefficients of panicle length, number of primary branches per panicle, density of primary branches, and grain density per panicle are shown in Table 3.1. Except for a certain degree of negative correlation between panicle length and density of primary branches, there was a significant positive correlation between other traits. Among them, the correlation coefficient between grain number per panicle and grain density per panicle was the highest, 0.914. The correlation between the density of density of primary branches and grain number per panicle was the lowest, being 0.355.

FIG. 8 shows frequency distribution of five panicle traits in the BC₃F₂-LQ96 population.

The black and white arrows in the figure indicate the mean (M1 and M2) of Kongyu 131 and the population, respectively. The phenotypic value of Kongyu 131 is from the mean of 10 Kongyu 131, and the phenotype of BC₃F₂-LQ96 population is the mean of 96 plants within the population.

TABLE 4
Correlation coefficients between five panicle traits in
BC₃F₂-LQ96 population

| | PL | NPB | DPB | GNP |
|---|---|---|---|---|
| NPB | 0.598** | | | |
| DPB | −0.059 | 0.763** | | |
| GNP | 0.768** | 0.777** | 0.355* | |
| GDP | 0.459** | 0.721** | 0.533** | 0.914** |

* and ** indicate that the correlation is significant at the level of p < 0.05 and p < 0.01.

2.1.5 Genetic Analysis of Genotypes and Phenotypes

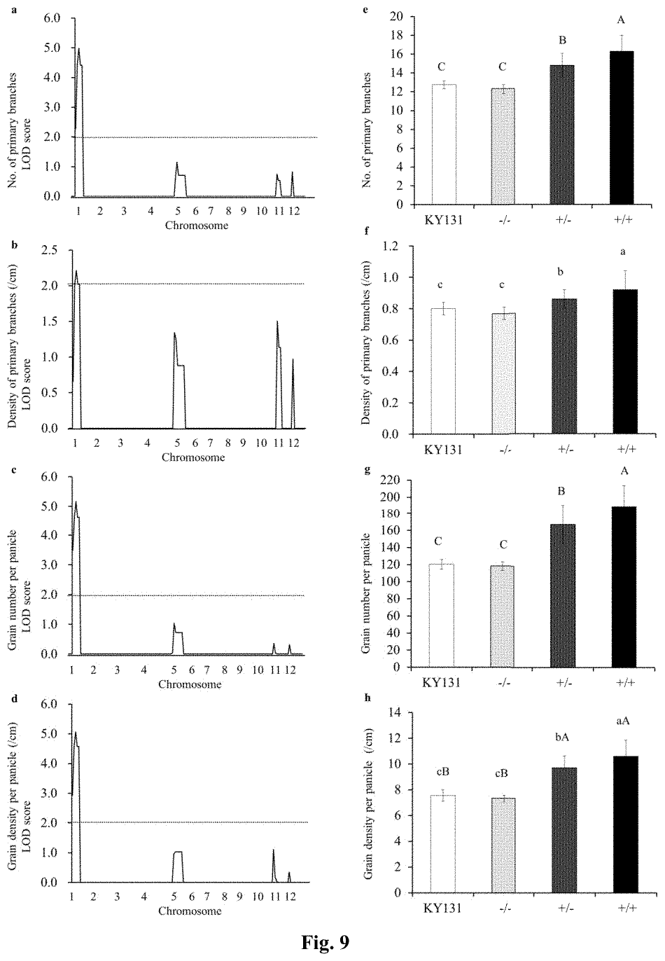

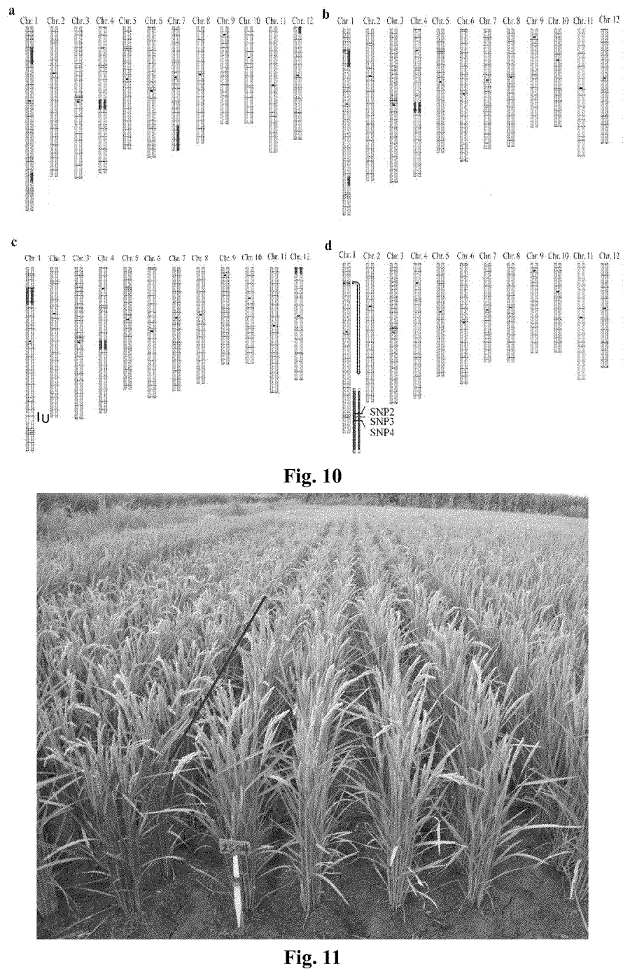

Genetic analysis was carried out in terms of the BC₃F₂-LQ96 population genotype and phenotype, and the results showed that the chromosome fragment located on chromosome 1 simultaneously had significant effects on number of primary branches per panicle (NPB), density of primary branches (DPB), grain number per panicle (GNP), and grain density per panicle (GDP) (FIG. 9a˜d). Confidence intervals SNP1˜SNP5 contain Gn1a locus, in which the LOD value of DPB is 2.2, and the LOD values of other 3 traits are 5.0˜5.2, which can explain 39.9%, 20.3%, 41.1% and 40.4% phenotypic variation, respectively. The same locus was detected for all four traits, which was consistent with a significant positive correlation between these traits. Gn1a from the donor GKBR was synergistic, with additive effect values of 2.0, 0.08, 34.95, and 1.65, respectively, showing partial dominance (Table 5).

TABLE 5
QTLs of panicle-related traits detected in
BC₃F₂-LQ96 population and their effects

| | | Marker | Additive | Dominant | | |
|---|---|---|---|---|---|---|
| Traits | Chr | interval | effect | effect | LOD | Var. % |
| NPB | 1 | SNP1~SNP5 | 2.00 | 0.50 | 5.0 | 39.9% |
| DPB | 1 | SNP1~SNP5 | 0.08 | 0.02 | 2.2 | 20.3% |
| GNP | 1 | SNP1~SNP5 | 34.95 | 14.05 | 5.2 | 41.1% |
| GDP | 1 | SNP1~SNP5 | 1.65 | 0.74 | 5.1 | 40.4% |

The additive effect and the dominant effect and LOD values in the table are all calculated for the effect of SNP3 locus, and Var. % indicates the phenotypic variation rate explained by the QTL.

Comparison of the phenotypic values of the panicles of Kongyu 131, of three different genotypes (homozygous Kongyu 131, heterozygous and homozygous GKBR) at Gn1a loci and of the control Kongyu 131 found: The average value of number of primary branches per panicle (NPB), density of primary branches (DPB), grain number per panicle (GNP), and grain density per panicle (GDP) of homozygous GKBR and heterozygous plants were significant increased than that of Kongyu 131 and homozygous Kongyu 131 plants. There was no significant difference between homogeneous Kongyu 131 and Kongyu 131. In addition, the phenotypic value of homozygous plants with Gn1a is significantly higher than that of heterozygous plants. (FIG. 9e˜h). The above results indicated to that the introgressing of favorable Gn1a allelic variation of the donor GKBR significantly increased the number of primary branches per panicle (NPB), density of primary branches (DPB), grain number per panicle (GNP), and grain density per panicle (GDP) of Kongyu 131. At the same time, there was no significant difference in the panicle length between the three different genotypes at Gn1a and Kongyu 131, so it was indicated that the increase in the grain number per panicle (GNP) was mainly due to the increase in the number of primary branches rather than the increase in the panicle length. That is, the density of the panicle increases the grain number per panicle.

FIG. 9 shows genetic analysis of the genotype and phenotype of the BC₃F₂-LQ96 population. (a-d) LOD values for four panicle related traits. The abscissa indicates 160 SNP markers on 12 pairs of chromosomes. (e-h) Comparison of phenotypic values of four panicle related traits of three genotypes at the Gn1a locus in the BC₃F₂-LQ96 population. KY131 represents Kongyu 131, −/− represents homozygous Kongyu 131 at Gn1a locus, +/− represents heterozygous type at Gn1a locus, and +/+ represents homozygous GKBR type at Gn1a locus. A, B, C, and a, b, and c above the bar graph represents significant differences at the levels of p≤0.01 and p≤0.05, respectively, by the T test (FIG. 9).

2.1.6 Recombinant Selection, Background Selection and Evaluation of Selected Plants

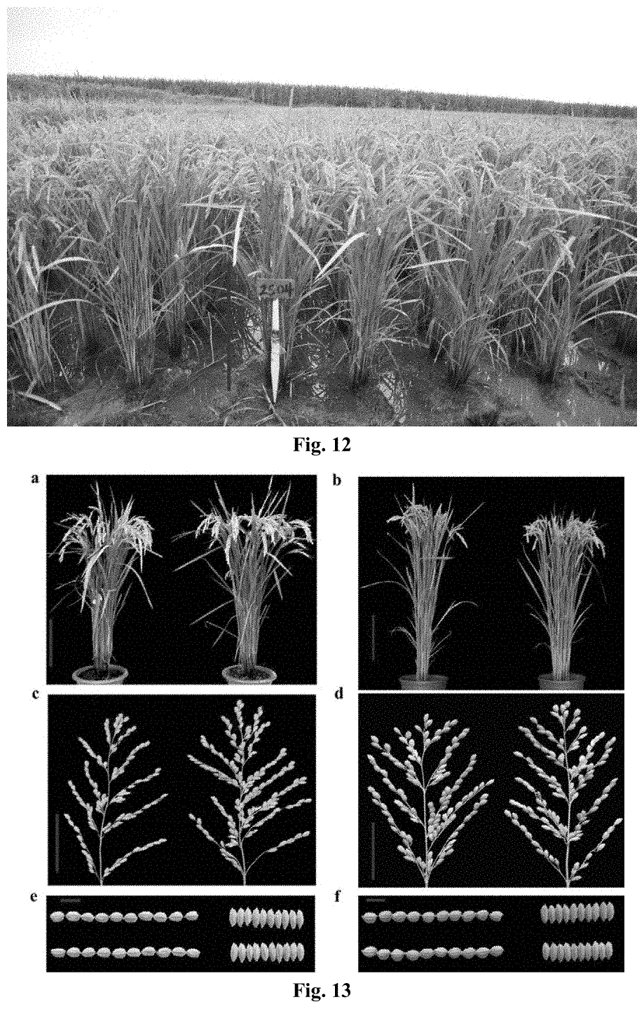

First crossover and selection: to shorten the introgressed target chromosome fragment and to minimize linkage drag, 90 recombinant lines with crossovers between SNP1 and SNP5 were selected from 960 BC₃F₂ plants derived from BC₃F₁-22F01. Then, the markers heterozygous for BC₃F₁-22F01 and 60 newly added markers located in the large interval without markers on chromosome were used to detect the 90 recombinant plants, from which a plant named BC₃F₂-2B09 containing to the Gn1a chromosome fragment, and with crossovers between SNP1 and SNP2 upstream of Gn1a and containing the minimum non-target chromosome fragment, was selected (FIG. 10a) The plant was detected with 220 SNP markers with an average distance between the markers of 1.7-Mb, the introgressed target fragment is approximately 4-Mb, and one non-target chromosome fragment is observed on chromosomes 1, 4, 7 and 12, respectively. Among them, the non-target chromosome fragments located on chromosomes 1, 4 and 7 were re-detected after the markers were added, and the background recovery ratio was 97.49%.

The second crossover and selection: to shorten the length of the introgressed target chromosome fragment, 20 plants with crossovers between SNP4 and SNP5 were selected according to the genotype of SNP4 and SNP 5 from 1240 plants in BC₃F₃ population derived from BC₃F₂-2B09 selfing. Then, the markers heterozygous for BC₃F₂-2B09 were used to detect the 20 recombinant plants, from which a line named BC₃F₃-652E09 containing the Gn1a chromosome fragment and the minimum non-target chromosome fragments (FIG. 10b). The introgressed target fragment of this plant is approximately 430-Kb, and one non-target fragment was observed on chromosomes 1 and 4, respectively, and the background recovery ratio was 98.45%.

In addition, in the progeny BC₃F₃ population derived from BC₃F₂-2B09 self-crossing, BC3F3-624A05 was selected according to the genotype of 220 SNP markers (FIG. 10c), and the plant was homozygous for the GKBR type at the locus containing the Gn1a chromosome fragment (SNP2˜SNP5). In addition, one non-target chromosome fragment was introgressed on chromosomes 4 and 12, respectively. The plant was planted in Changchun and Jiamusi in 2016 for the evaluation of the comprehensive phenotypic traits of the improved lines.

Purification and fixation of selected plants: in order to further exclude non-target chromosomal fragments on chromosomes 1 and 4 and obtain a new Kongyu 131 genome containing only a small chromosome fragment of homozygous Gn1a, BC₃F₃-652E09 obtained from the second crossover and selection was backcrossed with Kongyu 131, and the BC₄F₁-255A01 plant was selected among the progeny, and then BC₄F₁-255A01 was self-crossed to obtain 288 progenies from which 3 homozygous BC₄F₂-350A09 plants containing only small target chromosome fragment were selected. The background recovery ratio was 99.89%.

FIG. 10 shows graphical genotype (GGT) of selected plants

(a) BC₃F₂-2B09; (b) BC₃F₃-652E09; (c) BC₃F₃-624A05; (d) BC₄F₂-350A09. In the figure, the gray bar represents the chromosome fragment derived from KongYu 131, the black bar represents the chromosome fragment derived from the donor GKBR, and the white column represents that the chromosome fragment type is unknown (the genotype is not determined), the thin black horizontal line represents the position of the SNP marks, and the thick black horizontal line represents the centromere.

2.1.7 Comparison of Agronomic Traits Between Kongyu 131 and its Improved Lines

In 2016, Jiamusi, the field performance of Kongyu 131 and its improved line BC₃F₃-624A05 in the filling and maturity stages are shown in FIG. 11 and FIG. 12. The plant type, panicle type and grain type of the Kongyu 131 and its improved line BC₃F₃-624A05 and the finally constructed single point substitution line BC₄F₂-350A09 are shown in FIG. 13, and the agronomic traits in Changchun in 2016, Jiamusi in 2016 and Jiamusi in 2017 are compared as shown in Table 6.

FIG. 11 shows field display of Kongyu 131 and its improved line BC₃F₃-624A05 at the filling stage (Jiamusi, 2016). The left side of the red line is the Kongyu 131, and the right side is the improved line BC₃F₃-624A05.

2.1.7.1 the Improved Line is Cultivated in Jiamusi, and there is No Significant Difference in Heading Stage.

In the field trials in 2016 and 2017, seeds were soaked on April 10, and the heading days of the improved lines BC₃F₃-624A05, BC₄F₂-350A09 and Kongyu 131 was not significantly different in Jiamusi, all of which were 103 days; however, in Changchun, the heading date of the improved line is 4 days later than Kongyu 131, and it is speculated that this difference may be affected by the environment, such as the water temperature of irrigation and the influence of fertilizer.

FIG. 12 shows field display of the Kongyu 131 and its improved line BC₃F₃-624A05 at maturity (Jiamusi, 2016). The left side of the red line is the Kongyu 131, and the right side is the improved line BC₃F₃-624A05.

2.1.7.2 the Plant Height of the Improved Line B C₃F₃-624A05 Increased, and the Plant Height of BC₄F₂-350A09 Showed No Significant Difference.

The plant height of the improved line BC₃F₃-624A05 was increased by 4 cm and 5.4 cm relative to Kongyu 131 in the two test sites of Changchun and Jiamusi respectively, and it is speculated that there are three reasons for this difference in plant height: first, the multi-effect of Gn1a itself; second, the role of other loci near Gn1a; third, possibly the undetected donor chromosome fragments on the genetic background of the improved line. Further field trials of single point substitution line BC₄F₂-350A09 showed that the plant height of BC₄F₂-350A09 was not significantly different from that of Kongyu 131, thus eliminating the plant height increase of improved line BC₃F₃-624A05 due to multi-effect of Gn1a.

2.1.7.3 the Yield Traits of the Improved Line, Such as the Number of Branches and the Grain Number Per Panicle, Increased Significantly

The number of primary branches per panicle (NPB), density of primary branches (DPB), grain number per panicle (GNP) and grain density per panicle (GDP) of the improved lines BC₃F₃-624A05 and BC₄F₂-350A09 were significantly increased in the three different environments than Kongyu 131. Although the panicle length (PL) increased but did not reach a significant level, indicating that the increase of the yield per plant of the improved line was mainly caused by that the number of primary branches per panicle and the grain number per panicle increased so that the density of the grains increased.

2.1.7.4 the Thousand Grain Weight Drop of the Improved Line

At the two test sites in Changchun and Jiamusi, the thousand grain weight (TGW) of the improved line BC₃F₃-624A05 was reduced by 1.3 g and 2.0 g, respectively. The thousand grain weight (TGW) of the improved line BC₄F₂-350A09 was also reduced by 0.8 g compared to the Kongyu 131. Further analysis found that compared with Kongyu 131, the grain length (GL), grain width (GW) and grain thickness (GT) of BC₃F₃-624A05 and BC₄F₂-350A09 were slightly smaller than that of the control Kongyu 131 in three environments, which was consistent with the drop of thousand grain weight of the improved line BC₃F₃-624A05 and the single point substitution line BC₄F₂-350A09 compared to the control Kongyu 131.

2.1.7.5 the Yield Per Plant of the Improved Line is Significantly Increased

In Changchun in 2016, the yield per plant (YP) of the improved line BC₃F₃-624A05 was 4.7 g higher than that of Kongyu 131, an increase of 8.3%; in Jiamusi in 2016, the yield per plant (YP) of the improved line BC₃F₃-624A05 was increased by 6.9 g in Jiamusi, an increase of 11.9%. The actual yield per plot (AYP) of the improved line BC₃F₃-624A05 at two plots increased by 7.8% and 10.1%, respectively, compared with the control Kongyu 131. Compared with Jiamusi, the increase in yield per plant (YP) of the improved line in Changchun was less likely due to less increase in grain number per panicle (GNP) and lower seed setting percentage (SSP) than Kongyu 131. However, the increase in the grain number per panicle (GNP) in the improved line in Changchun was less than that in Jiamusi, which may be due to about 10 days shorter of the heading date of the improved line in Changchun. In 2017, in Jiamusi, the yield per plant (YP) of the improved line BC₄F₂-350A09 increased by 3.5 g, an increase of 6.6%. The increment in yield per plant (YP) was lower than that of BC₃F₃-624A05 in 2016 was likely due to a decrease in the increment.

2.1.8 there is No Significant Difference in Quality Traits Between the Improved Line and the Kongyu 131

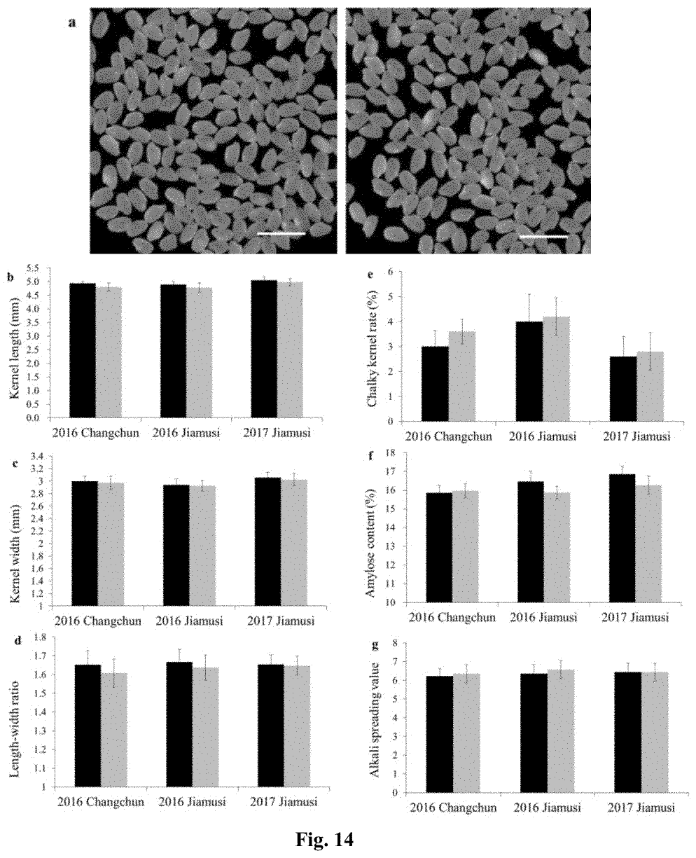

The appearance quality and the amylose content and alkali spreading value in cooking quality of milled rice from the improved line and the control Kongyu 131 are shown in FIG. 14. It can be seen that the transparency of the improved line BC₄F₂-350A09 was not significantly different from that of the Kongyu 131 (FIG. 14a); in the three different environments of Changchun in 2016, Jiamusi in 2016 and Jiamusi in 2017, the kernel length (KL), kernel width (KW) and the length-width ratio (LWR) of the improved line were slightly smaller than the control Kongyu 131, but did not reach a significant level (FIG. 14b-d). Similarly, the chalky kernel rate (CKR) in the three environments was slightly higher than that of the control Kongyu 131, but the difference was not significant (FIG. 14e). The amylose content (AC) and alkali spreading value (ASV) of the improved line were also not significantly different from those of Kongyu 131 (FIG. 14f, g).

FIG. 13 shows plant type, panicle type and grain type of Kongyu 131 and its improved lines BC₃F₃-624A05 and BC₄F₂-350A09. (a) Plant type, Kongyu 131 (left), BC₃F₃-624A05 (right), scale bar 20 cm. (b) Plant type, Kongyu 131 (left), BC₄F₂-350A09 (right), scale bar 20 cm. (c) Panicle type, Kongyu 131 (left), BC₃F₃-624A05 (right), scale bar 5 cm. (d) Panicle type, Kongyu 131 (left), BC₄F₂-350A09 (right), scale bar 5 cm. (e) Grain type, Kongyu 131 (top), BC₃F₃-624A05 (bottom), scale 1 cm. (f) Grain type, Kongyu 131 (top), BC₄F₂-350A09 (bottom), scale bar 1 cm. (a), (c), (e) 2016 Changchun; (b), (d), (f) 2017 Jiamusi.

TABLE 6
Comparison of agronomic traits of Kongyu 131 and its improved lines BC₃F₃-624A05 and BC₄F₂-350A09

| | 2016 Changchun (E125°18′, | | | | | |
|---|---|---|---|---|---|---|
| | N44°43′) | | 2016 Jiamusi (E130°57′, N46°23′) | | 2017 Jiamusi (E130°57′, N46°23′) | |
| Traits | KY131 | BC₃F₃-624A05 | KY131 | BC₃F₃-624A05 | KY131 | BC₄F₂-350A09 |
| DTH | 93.2 ± 0.8 | 97.2 ± 1.5** | 103.6 ± 0.8 | 103.1 ± 0.7 | 103.3 ± 1.4 | 102.8 ± 2.2 |
| PH (cm) | 70.7 ± 2.2 | 74.7 ± 1.1** | 70.6 ± 1.8 | 76.0 ± 3.1** | 64.0 ± 3.5 | 62.1 ± 2.3 |
| ETP | 27.3 ± 3.3 | 27.1 ± 3.1 | 31.5 ± 3.6 | 31.4 ± 2.4 | 34.7 ± 3.5 | 35.1 ± 3.5 |
| PL (cm) | 16.6 ± 0.5 | 17.4 ± 0.5 | 16.5 ± 0.9 | 17.2 ± 0.4 | 16.1 ± 0.6 | 16.1 ± 0.4 |
| NPB | 11.8 ± 0.6 | 14.7 ± 0.6** | 12.2 ± 1.0 | 14.9 ± 0.5** | 10.2 ± 0.9 | 12.6 ± 0.8** |
| DPB (/cm) | 0.71 ± 0.04 | 0.85 ± 0.06** | 0.75 ± 0.09 | 0.86 ± 0.02** | 0.63 ± 0.05 | 0.78 ± 0.05** |
| GNP | 119.3 ± 8.0 | 168.1 ± 2.6** | 123.4 ± 8.3 | 186.2 ± 17.8** | 116.3 ± 8.1 | 136.6 ± 7.5** |
| GDP (/cm) | 7.18 ± 0.45 | 9.79 ± 0.11** | 7.50 ± 0.38 | 10.69 ± 0.79** | 7.2 ± 0.4 | 8.5 ± 0.5** |
| SSP (%) | 97.0 ± 1.7 | 93.8 ± 1.9** | 98.0 ± 0.3 | 97.4 ± 0.6 | 95.5 ± 1.6 | 94.8 ± 2.0 |
| YP (g) | 56.4 ± 1.1 | 61.1 ± 1.6** | 57.8 ± 1.1 | 64.7 ± 1.8** | 53.0 ± 2.3 | 56.5 ± 2.0** |
| TGW (g) | 27.6 ± 0.6 | 26.3 ± 0.6** | 27.8 ± 0.5 | 25.8 ± 0.3** | 27.2 ± 0.6 | 26.4 ± 0.7* |
| GL (mm) | 7.55 ± 0.08 | 7.52 ± 0.03 | 7.48 ± 0.11 | 7.36 ± 0.10 | 7.50 ± 0.08 | 7.46 ± 0.10 |
| GW (mm) | 3.69 ± 0.03 | 3.61 ± 0.06 | 3.64 ± 0.06 | 3.54 ± 0.03 | 3.73 ± 0.03 | 3.71 ± 0.04 |
| GT (mm) | 2.33 ± 0.00 | 2.28 ± 0.04** | 2.33 ± 0.02 | 2.30 ± 0.01* | 2.34 ± 0.03 | 2.31 ± 0.02* |
| AYP (Kg) | 4.38 ± 0.21 | 4.72 ± 0.26** | 4.55 ± 0.25 | 5.01 ± 0.35** | | |

The values in the table are mean ± standard deviation, and the phenotypic values of Kongyu 131, BC₃F₃-624A05 and BC₄F₂-350A09 are derived from the mean of 30 phenotypic values. The planting density is 30 cm × 20 cm, with one plant per hole during cultivation. The actual yield per plot (AYP) is derived from the mean of 10 plots, and the area of a single plot is 5.76 m² (2.4 m × 2.4 m).
*and **indicate significant differences at p ≤ 0.05 and p ≤ 0.01 according to the T test.

FIG. 14 shows comparison of grain quality traits between Kongyu 131 and its improved lines. (a) Grain appearance of milled rice, 2017 Jiamusi, Kongyu 131 is on the left, 2017 Jiamusi, improved line BC4F2-350A09 is on the right, scale bar is 1 cm; (b) polished rice length (n=30); (c) milled rice width (n=30); (d) aspect ratio (n=30); (e) chalky kernel rate (n=500); (f) amylose content (n=4); (g) alkali spreading value (n=14); In b-g, the black column represents Kongyu 131, and the gray column represents the improved line, in which the improved line BC₃F₃-624A05 for 2016 Changchun and 2017 Jiamusi, and the improved line BC₄F₂-350A09 for 2017 Jiamusi.

### EXAMPLE 2

Improving Kongyu 131 by Updating the Grain Length Locus GS3 to Increase the Yield of Kongyu 131

The world's population continues to increase, and it is a great challenge to enhance the production of crops to supply the increasing demand continuously. Although traditional breeding methods have made a great contribution to solving human needs for food, these methods have problems such as large workload, unpredictability, and non-repetition. The present disclosure accurately updates GS3 locus of Kongyu 131 through the method of genome upgrading, and overcomes the problems of large workload, unpredictability, and non-repeatable in the traditional breeding methods. We use this method to improve the grain length locus GS3 of Kongyu 131. Single nucleotide polymorphism (SNP) marker primers between Kongyu 131 and donor BR were designed by genomic resequencing of Kongyu 131 and the donor BR; 219 pairs of markers were selected using high resolution dissolution curve analysis (HRM) to screen the designed primers, and then selection was carried out against the whole genome; At the same time, SNP1-SNP5 were designed based on the sequence polymorphism between the two parents at the upstream and downstream of the GS3 locus to select target locus GS3 and minimize linkage drag. We started from BC3F1 and selected a plant, named improved line, in which segment of GS3 locus with a length less than 117 kb is from donor BR and the background recovery ratio was 99.55%. The field cultivation trials in Jiamusi showed that after improvement, the improved line with grain length locus GS3 was significantly increased in grain length and hundred grain weight compared with Kongyu 131, and the yield was also greatly increased. This proves that this is an effective breeding method and is expected to become one of the main methods for future breeding and will play an important role in solving the food problem.

Keywords: Kongyu 131, GS3, SNP, HRM

### Introduction

As the world's population continues to increase, the total food demand is growing. However, how to continuously and effectively increase food production is a severe challenge. On the one hand, the arable land is decreasing with the development of urbanization; on the other hand, the production of crop is influenced inevitably by some environment factors such as global warming (Takeda and Matsuoka 2008). In the past few decades, global food production has increased significantly twice. One is the application of semi-dwarf genes in wheat and rice, namely the “green revolution” (A. Sasaki et al. 2002; Jinrong Peng et al. 1999; Spielmeyer et al. 2002); the other one is the cultivation and production of hybrid rice in China and Southeast Asian countries in the 1970s (Shi-Hua Cheng and Ye-Yang Fan 2007). However, studies have shown that food production has increased slowly in some areas in recent years, and even some areas have reduced food production (Ray et al. 2012). Therefore, how to continuously and effectively increase food production to meet increasing demand is an urgent problem to be solved in the future. In the case of a gradual decrease in the area of arable land, increasing the yield per unit area by improving crops is an effective way.

The traditional way of improving crops, that is, traditional breeding, relies on the experience of breeders to select plants that are considered to be good in the field, and then carry out continuous field screening, and finally obtain good plants and cultivate same as new varieties. However, this method of using experience to select in the field has disadvantages such as unpredictable, non-repeatable and large workload.

With the discovery and utilization of molecular markers, breeders can use molecular markers that are closely linked to good traits to aid in the selection of plants, i.e., molecular markers-assisted selection (MAS (Knapp 1998)). Molecular markers-assisted selection has indeed brought great convenience to breeders, not only reducing a large amount of field selection work, but also improving the accuracy of selecting target plants. However, the MAS markers commonly used by breeders are only closely linked to the target traits, which inevitably lead to the inconsistency between the selected plants and the expectation, i.e., there are crossovers between the target traits and the markers (Andersen and Lubberstedt 2003). Similarly, this method only focuses on the selection of target traits, without considering the information elsewhere in the genome, so whenever there is a problem with the bred varieties, the cause cannot be found. Therefore, this breeding method still has problems such as poor predictability and poor repeatability.

At present, the rapid development of genome sequencing, the discovery of a large number of functional genes and the in-depth study of gene regulation mechanisms (Huang et al. 2013; James et al. 2003; Miura et al. 2011; Sakamoto and Matsuoka 2008; Wang and Li 2008, 2011; Xing and Zhang 2010; Zhou et al. 2013; Zuo and Li 2013), provide breeders with a wealth of information available, to some extent allowing breeders to design breeding according to their breeding goals. In 2003, two Israeli scientists proposed the concept of design breeding (Peleman and van der Voort 2003). Future breeders are expected to use the existing genome sequencing information, functional gene information and large amount of other research results, combine genomic information according to different breeding goals, and cultivate crop varieties with high yield, high quality, stress resistance and other good traits to provide a powerful approach for solving food issues.

This research is based on the rapid development of genomic information, the extensive discovery and research of functional genes, and the extensive use of digital information and is for solving the problems in traditional breeding methods such as large workload in the field, long breeding period, unpredictable and non-predictable breeding results, this study proposes a method of upgrading breeding, which has the following features compares with traditional breeding methods: (1) The method can complete almost all the selection work in the laboratory, that is, the selection work is mainly done indoors by SNP genotype analysis, does not require a large number of field screening, which greatly reduces the workload of field selection; (2) The method can accurately select the cause genes of the target traits, can predict the breeding results, that is, has high predictability; (3) This method can not only select the cause genes of the target traits, but also can select against the whole genome. When there is a problem with the bred varieties, the cause of the problem can be immediately found, that is, it has stability and repeatability.

Rice is a major food crop, with more than half of the world's population taking rice as main food. At the same time, rice is a monocotyledonous model plant because rice which has a small genome and complete genomic information and a large number of available resources. It is generally believed that the yield per plant of rice depends on the panicle number per plant, grain number per panicle, grain weight and filling (Sakamoto and Matsuoka 2008; Xing and Zhang 2010). Grain weight is one of the key factors in rice production, so increasing grain weight by improving crops is one of the most effective ways to increase yield. In the past few decades, a large number of functional genes related to rice yield have been discovered and studied (Huang et al. 2013; Miura et al. 2011; Wang and Li 2011; Xing and Zhang 2010; Zuo and Li 2013), GS3 is the first grain type gene found in rice (Fan et al. 2006; Mao et al. 2010).

Therefore, in this study, the grain length of the main rice variety Kongyu 131 was improved. Through the genome sequencing information of donor BR and Kongyu 131, SNP markers covering the whole genome were designed, and SNP1-SNP5 were designed was designed for the GS3 locus to select target genes. Continuous selection was started from BC3F1 and a plant, named improved line, in which segment of target GS3 locus with a length less than 117 kb was from donor BR and the background recovery ratio was 99.55% Through the field cultivation in Jiamusi in the summer of 2016, the investigation of agronomic traits of the improved line and Kongyu 131 found that the grain length and hundred grain weight of the improved line were significantly improved, and the yield was also greatly improved. Therefore, this method is very effective in improving the GS3 locus of Kongyu 131, and has achieved the expected goal, which proves that the method is controllable, predictable, repeatable, and the workload is greatly reduced. Our trial results show that this method is a very effective breeding method and provides a new breakthrough for future breeding methods.

Materials and Methods

Parent and Material Construction

In this experiment, the short-grained japonica rice variety Kongyu 131 was used as the recurrent parent. This variety was once the main plant variety in Heilongjiang Province, and it has the characteristics of early maturity, high yield and low temperature tolerance. The donor BR is a long-grained indica variety. We used Kongyu 131 as the background, crossed with donor BR to obtain F1, and then backcrossed with Kongyu 131 three times to obtain a BC3F1 polulation containing a total of 137 lines. Starting from BC3F1, the target plants were selected by molecular markers analysis, and the target plants were continuously self-crossed to obtain the final improved line BC3F4.

TABLE 7
Phenotype of Kongyu 131 and improved line BC3F4

| | 2016 | | 2017 | |
|---|---|---|---|---|
| Traits | BC3F4-4 | KY 131 | BC3F4-4 | KY 131 |
| GL (mm) | 7.73 ± 0.14* | 6.90 ± 0.11 | 7.90 ± 0.13* | 6.94 ± 0.09 |
| GW (mm) | 3.59 ± 0.08 | 3.53 ± 0.05 | 3.47 ± 0.11 | 3.44 ± 0.03 |
| HGW (g) | 3.08 ± 0.04* | 2.65 ± 0.05 | 2.98 ± 0.05* | 2.73 ± 0.06 |
| TYP (g)^{a} | 50.27 ± 4.81 | 48.11 ± 7.7 | 48.82 ± 6.45 | 48.39 ± 5.87 |
| TYP (g)^{b} | 55.96 ± 11.16* | 40.82 ± 4.31 | — | — |
| TYP (g)^{c} | — | — | 48.52 ± 8.26 | 48.06 ± 3.79 |
| PNP | 27.4 ± 2.87* | 31.3 ± 3.16 | 24.13 ± 1.73* | 34 ± 3.38 |
| GNP | 125.8 ± 10.51 | 116.8 ± 23.5 | 126.5 ± 14.13 | 116.38 ± 8.42 |
| PH (cm) | 72.2 ± 2.82 | 71.45 ± 1.06 | 72.63 ± 0.58* | 67.60 ± 1.75 |
| PL (cm) | 17.7 ± 1.22 | 17.58 ± 1.74 | 16.28 ± 1.35 | 16.06 ± 0.63 |
| DTH | 107.25 ± 2.06 | 107.25 ± 2.63 | 102.50 ± 1.64 | 102.83 ± 2.14 |

Data presented as the means with standard deviations were obtained from plants in a randomized complete block design with three replications under natural conditions at Jiamusi in 2016 and 2017. The planting density was 30 cm×20 cm and one plant per hill GL Grain length, GW Grain width, HGW Hundred grain weight, TYP Total grain yield per plant, PNP panicle number per plant, GNP grain number of main panicle, PH plant height, PL main panicle length, DTH Days to heading * represents significance at p≤0.05 based on Student's t-tests, n=10 a The planting density was 30 cm×20 cm and one plant per hill b The planting density is 30 cm×20 cm and three to four plants per hill c The planting density is 30 cm×14 cm and three to four plants per hill; — indicates no data

Resequencing of Parent and Sequence Alignment of GS3 Locus Gene

We used the HiSeq2000 sequencer to re-sequence the genome of Kongyu 131 and obtained the SNP information between the Kongyu 131 and BR. The GS3 gene sequence was downloaded from the NCBI database, and the GS3 gene sequence was aligned and analyzed by DNAMAN between the Kongyu 131 and BR.

SNP marker design and genotyping Using the SNP information between Kongyu 131 and BR obtained by resequencing, we designed SNP marker primers covering the whole genome, and selected 219 pairs of markers with polymorphism between the Kongyu 131 and the BR for genome-wide selection. According to the difference of GS3 gene and upstream and downstream sequences between Kongyu 131 and BR, five polymorphic SNP markers were designed and screened: SNP1-SNP5, in which SNP1 and SNP2 were located upstream of GS3 gene, and SNP4 and SNP5 were located downstream of GS3 and were mainly used to minimize linkage drag with GS3 gene; SNP3 was located in the GS3 gene, for the selection of target genes. Polymorphism verification analysis of SNP marker primers was performed using the HRM analysis method (Wittwer 2009).

TABLE 8
5 SNP markers for selection of target genes

| Markers | Chr. | Position | Forward primer | Reverse primer |
|---|---|---|---|---|
| SNP1 | 3 | 16,854,214 | TGGTACACAGCATATCATGGAAC | CAGAAGTGTTATAACTACATATTTGC |
| SNP2 | 3 | 17,296,672 | ATCTGCAACAAACAAGAGGATC | CTTGAGTTTCCACTCACAAACTTTTC |
| SNP3 | 3 | 17,369,402 | GAAACAGCTGGCTGGCTTACTCTC | GATCCACGCAGCCTCCAGATGC |
| SNP4 | 3 | 17,413,766 | TTAGGACATATCGGCGTGCGTTTA | CAGAGAAGCATCATTGAACGAACA |
| SNP5 | 3 | 17,874,647 | ATGCAACCTTTTCTCCCTTCCTAT | TCTAAAGGTTAACTCAGTAAAATCCT |

Selection of Target Plant (Improved Line)

In order to obtain plants with GS3 locus from donor BR and other loci in the genome from Kongyu 131, we first select plants with SNP3 as H-type, crossovers between SNP1 and SNP5 as much as possible, and high background recovery ratio in BC3F1. And then in the self-crossed progenies of the plants, target plants were selected through the following two consecutive selections:

First selection: in about 1,000 progenies (BC3F2) from self-crossing of the plant, plants with crossovers between the GS3 gene (SNP3) and the upstream marker SNP1 and SNP2 or and the downstream marker SNP4 and SNP5 were selected;

Second selection: in about 1,000 progenies (BC3F3) from self-crossing of the selected plant of the first selection, the plants with crossovers between the GS3 gene (SNP3) and the molecular markers (SNP1, SNP2 or SNP4, SNP5) on the other end were selected;

Thus the plants with crossovers between the GS3 gene (SNP3) and both the upstream (SNP1 or SNP2) and downstream (SNP4 or SNP5) were selected and self-crossed to select a plant (BC3F4) with homozygous GS3 locus from the donor BR and with the highest background recovery ratio.

Field Cultivation and Traits Investigation

In Jiamusi, the target plant (improved line) and Kongyu 131 was cultivated in a plot in a manner of 8*12, and managed according to the general rice cultivation method. After maturity, the traits of grain length, grain width, plant height, panicle length, number of primary branches per panicle, and hundred grain weight were investigated, and the total grain weight per plant was measured.

In addition, in order to compare the yield difference between the improved line and the Kongyu 131 in the field cultivation, we cultivated the primary improved line with the grain length locs improved and Kongyu 131 in the Jiamusi field according to the normal rice cultivation method, with each 1 mu, and the yield was measured. The primary improved line mentioned here is an improved line that the target locus GS3 has been improved, but the other loci in the genome are not completely from Kongyu 131. We measure the yield of 10 selected plants, and the selection method of the plants is based on the survival of all 8 plants around the plants to be selected, so as to ensure that the growth of the measured plants is affected by the surrounding environment as little as possible, and the measurement error is minimized.

Results

Kongyu 131 and BR have only one base variation in the coding region of GS3 gene, and GS3 gene was mapped between SNP1-SNP5.

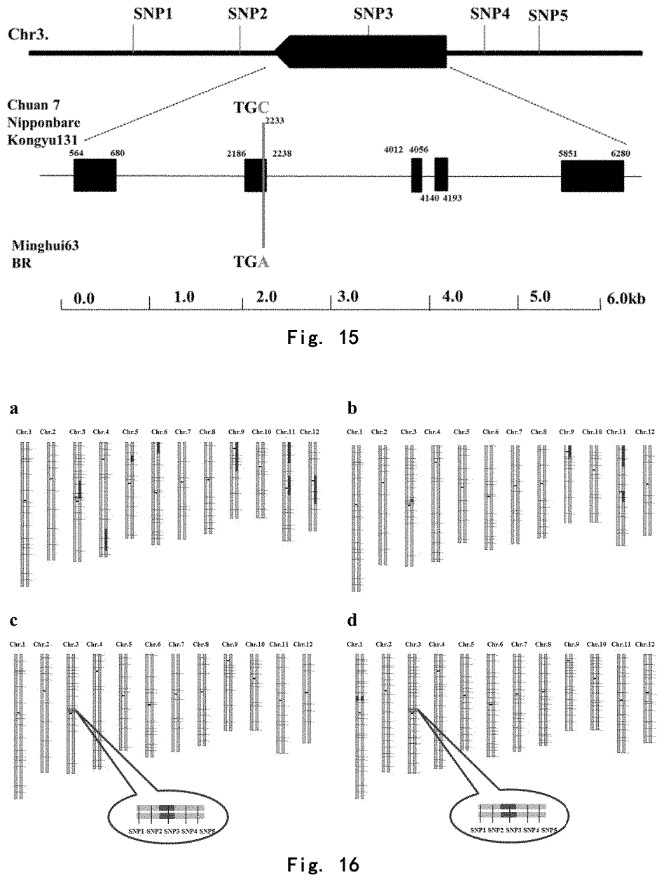

Sequence alignment analysis revealed that Kongyu 131 and BR were different in the 2233th base of the second exon of GS3 gene, and the base of Kongyu 131 was C at this position, and the base of BR was A at this position (FIG. 15). This is consistent with previous report of the base variation between the parents in cloned GS3 gene, that is, the short-grained variety Chuan7 and the long-grain variety Minghui63 are different in the second exon region of GS3 gene due to C-A base variation, causing premature termination of the transcription of the coding sequence of the long-grained variety, thus functional proteins can not be synthesized (Fan et al. 2006; Mao et al. 2010). At the same time, in order to accurately select GS3 gene, we designed SNP3 in GS3 gene based on the difference in sequence of GS3 gene between Kongyu 131 and BR. In addition, in order to shorten the length of the introgressed chromosome fragment as much as possible, and to exclude the fragment linked to the GS3 gene, SNP1, SNP2, SNP4, and SNP5 were designed upstream and downstream of GS3 gene, and SNP1 and SNP5 were separated by about 1M.

FIG. 15 GS3 locus sequence comparison between kongyu 131 and BR and SNP Markers used to selecting for gs3 gene from donor. kongyu 131 and BR have a base difference at the second exon as same as difference between Chuan7 and Minghui63 from which the GS3 gene was first cloned.

The GS3 Locus Fragment in the Improved Line is from the BR and is about 117 kb, and the Background Recovery Ratio is 99.55%.

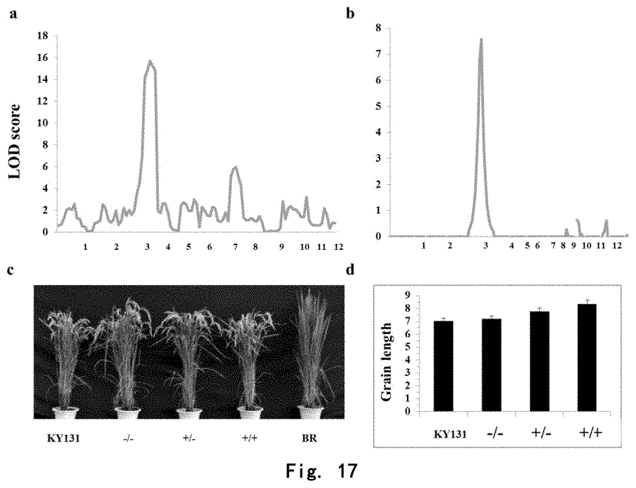

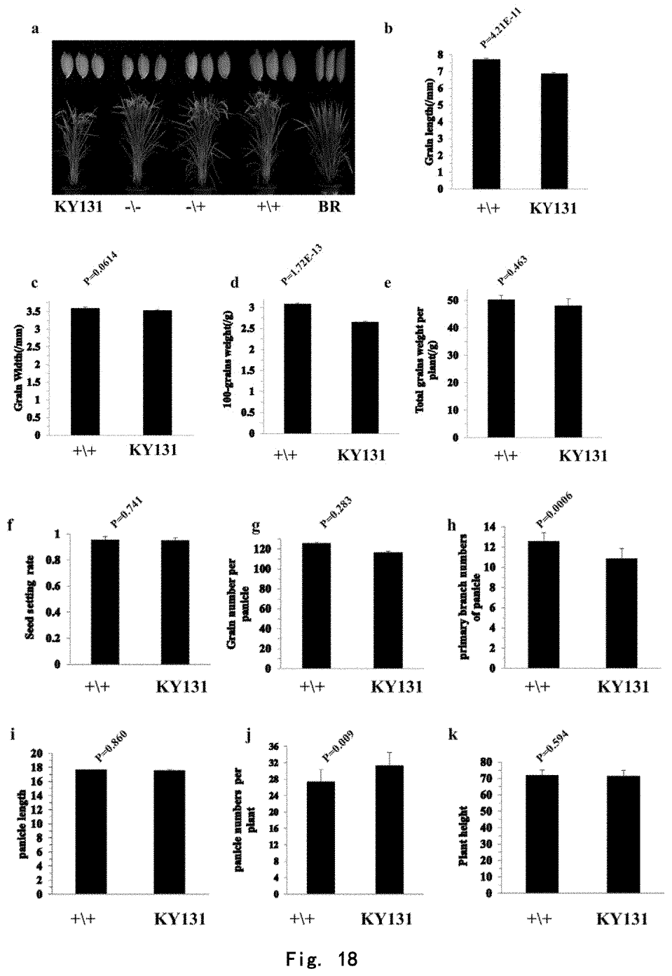

In order to select the plants with smallest fragment of GS3 locus from the donor BR, first, we selected 25 plants with H-type at the SNP3 locus among 137 plants in the BC3F1 population, 8 of which had crossovers between SNP1 and SNP3 or SNP3 and SNP5. And we selected one plant from the 8 plants with the highest background recovery ratio as the candidate plant for further selection. The plant was named as BC3F1-1 (FIG. 16a), and had crossovers between SNP3 and SNP5. Then, from the 960 plants of the self-crossing progeny BC3F2 of the plant, a plant having crossovers between SNP3 and SNP4 were selected and named as BC3F2-2 (FIG. 16B); then, in order to further narrow the fragment containing GS3, plants having crossovers between SNP3 and SNP1 or SNP2 were selected. We further selected the self-crossing progeny of BC3F2-2. Fortunately, we selected one plant from 400 self-crossing progenies having crossovers between SNP3 and SNP2, named as BC3F3-3 (FIG. 16c); finally, in order to select plants with the highest background recovery ratio, we performed genome-wide selection in the progenies of BC3F3-3 using 219 SNP markers covering the whole genome and with polymorphism between Kongyu 131 and BR, and we selected one target plant, named BC3F4-4, with homozygous GS3 locus of about 117 kb from the donor BR and with a background recovery ratio of 99.55% (FIG. 16d).

FIG. 16 Graphical Genotype (GGT) of selected individuals or lines. a BC3F1-1. b BC3F2-2. c BC3F3-3. d BC3F4-4. The green type means the chromosome of kongyu131, and the purple represents the fragments from BR.

QTL Analysis Confirmed that GS3 Allele Derived from Donor BR could Indeed Improve the Grain Length of Kongyu 131

In order to confirm that the GS3 gene from donor BR can improve the grain length of Kongyu 131, Kongyu 131 was used as the background, crossed with donor BR and self-crossed to obtain F2 population and BC3F2 population in which traits such as grain length were measured. The relationship between grain length and markers was analyzed by genotype analysis using Mapmaker/QTL 1.1b. A grain length locus (FIG. 17A˜b) was detected on chromosome 3, presumably the location of GS3 gene. Therefore, the grain length of Kongyu 131 can be improved by introgressing the GS3 allele from the donor BR (FIG. 17d).

FIG. 17 QTL analysis indicates that GS3 allelic from donor BR is positively increase grain length in recurrent parent of kongyu 131 background. a QTL analysis with F2 populations. b QTL analysis with BC3F2 populations. c The morphological feature of plant with different genotype at GS3 locus. d The grain length is significantly increased with the donor BR allelic at GS3 locus.

The Grain Length and Hundred Grain Weight of the Improved Line increased significantly.

In May 2016, we cultivated 96 plants from the selected improved line BC3F4-4 and Kongyu 131, respectively, in Jiamusi according to the 8*12 cultivation method at the same time. After maturity, the traits such as grain length, grain width, hundred grain weight, plant height, main panicle length, number of primary branches per panicle, and total grain weight per plant were investigated. Compared with Kongyu 131, the grain length and hundred grain weight of the improved line BC3F4-4 were increased significantly (FIG. 18b, d); the total grain weight per plant was increased, but not significant (FIG. 18e). At the same time, we found that the number of primary branches per panicle of the main panicle of the improved line was increased significantly (FIG. 18h), but the panicle number per plant was significantly reduced (FIG. 18j). This explains to some extent why the improved plant had no significant increase in the total grain weight per plant when the hundred grain weight was increased. Although it is still unclear what caused the increase in number of primary branches per panicle of the main panicle of the improved line and the decrease in the panicle number per plant at the same time, but this did not affect our improvement of grain length. We saw the grain length and hundred grain weight of the improved line were significantly increased compared to Kongyu 131 (FIG. 18b, d).

FIG. 18 Grain length and 100-grains weight significantly increased in improved line at GS3 locus compared with kongyu131. a The morphological feature of grain size and plant with different genotype at GS3 locus. b˜e Comparison of grain related traits. grain length and 100-grains weight significantly increased, while grain width and total grain weight per plant increased non-significant. f-j Comparison of main panicle related traits. Primary branch numbers of main panicle increased significantly while panicle numbers per plant decreased greatly and others varied little. k Plant height varied little.

Significant Increase in Total Grain Weight Per Plant in Primary Improved Line

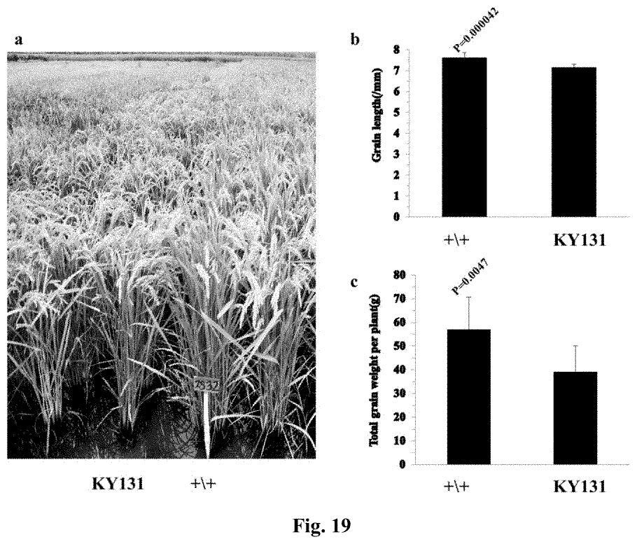

We selected 10 plants from Kongyu 131 and primary improved line cultivated in Jiamusi, respectively, for measuring the traits such as grain length and total grain weight per plant. It was found that the grain length and the total grain weight per plant of the primary improved line were significantly increased (FIG. 19b˜c). In addition, we found that the heading date of the primary improved line was on average 10 days later than Kongyu 131, and the plant height was 10 cm higher than Kongyu 131. This explains to some extent why the total grain weight per plant of the primary improved line was significant higher than that of Kongyu 131 but the increase in the total grain weight per plant of the improved line was not significant. Using QTL analysis of the genotypes and heading traits of the primary improved line, we found that there was a locus related to late heading on the chromosome 9 at about 9M, so we speculated that this locus led to the heading date of the primary improved line later than Kongyu 131 and final led to a significant increase in the total grain weight per plant of the primary improved line. Our QTL analysis confirmation and functional verification of the late heading locus are in progress. Next, we will improve and aggregate the late heading locus and GS3 locus of Kongyu 131 at the same time. The above trial results show that it is necessary to ensure sufficient supply of the source while improving the grain length, that is, increasing the background variety library. Therefore, it is extremely necessary to improve and aggregate the GS3 locus and the late heading locus of Kongyu 131.

FIG. 19 Field plot trial demonstrates that the primary improved line with GS3 allelic of donor BR at the background of kongyu131 significantly increased grain length and yield compared with the recurrent parent of kongyu131. a Field picture of kongyu131 and the primary improved line. b˜c Grain length and total grain weight per plant increased significantly of the primary improved line compare with kongyu 131. It strongly indicates that the improved line is better than the parent at grain length and yield by using the new method of update design breeding through update the grain length locus GS3.

### Discussion

We improved the grain length locus GS3 of Kongyu 131, and found that the grain length of the improved line was significantly increased by the field cultivation of the improved line. Therefore, this method is effective in the grain type improvement of Kongyu 131. And controllable and predictable improvement of target traits can be achieved completely by this method, Our process and results of improving the grain length locus GS3 of Kongyu 131 demonstrate that this method overcomes the shortcomings of large workload for field selection, unpredictable and non-repeatable results in traditional breeding. This method selects plants almost entirely indoors using SNP gene analysis, thus greatly reducing field workload. At the same time, the method uses the markers inside the gene to select the target traits, so there is no fear of loss of the target traits, and the selection process is precise and controllable. Furthermore, in addition to the selection of target traits, the method also selects other loci in the whole genome, which avoids the influence of other loci on the target traits, and does not change other good traits of the background variety. More importantly, when problems are found in the improved variety, the cause can be found in time and the same method can be used for improvement. Therefore, this breeding method is a very effective, precise and controllable breeding method, which is expected to play an important role in solving food security problems in the future.

Although the effect of improvement on the grain length of Kongyu 131 is very significant, it is based on the clear and thorough study of GS3 gene function and the reliable utilization of rice genome information. At the same time, we can see that there are some differences in the investigation results from the cultivation of the GS3 locus improved line and the cultivation of the primary improved line.

First, we compared the improved BC4F4-4 with Kongyu 131 cultivated in Jiamusi and found that the grain length and hundred grain weight were significantly increased, and the total grain weight per plant was increased but not significant. Among all the traits we investigated, it was found that the number of primary branches per panicle of the main panicle of the improved line was increased significantly, but the panicle number was decreased significantly. This explains to some extent the improved plant had no significant increase in the total grain weight per plant when the hundred grain weight was increased significantly. But we are still not completely sure what causes the number of primary branches per panicle of the main panicle of the improved line to increase significantly, and the panicle number to decreased significantly. We speculate that it may be related to the role of GS3, or it may be related to other loci in the genome, or it may be due to the effect of the cultivation environment. Therefore, we will further carry out cultivation trials on the improved lines to confirm the effects of GS3 on the traits of the improved lines.

Secondly, we compared the primary improved line with Kongyu 131 cultivated in Jiamusi and found that the the grain length and total grain weight per plant were increased significantly, at the same time found that the plant height was also increased by 10 cm on average, and the heading date was about 10 days later. We found through QTL analysis that the primary improved line contained a locus located on chromosome 1 that affected the heading date. We know that the growth period is one of the important factors affecting yield. Generally, the varieties with longer growth period will have higher yield, which explains why the total grain weight per plant of the primary improved line was significantly increased but the total grain weight per plant of the improved line was not increased significantly. We are performing further QTL analysis and verification of the lous affecting the heading date of the primary improved line. In the next step, we will simultaneously improve and aggregate the lous and the GS3 locus. According to the library-source relationship theory (Wang 2008), by simultaneously aggregating the late heading locus and the GS3 locus, this not only improves the grain length of Kongyu 131 (increased the library), but also provides sufficient source for the increased library.

Based on the above trials and analysis, we believe that to improve the variety by using upgrade design breeding, the following points must be met: 1. Reliable and accurate genomic information; 2. Large-scale discovery and functional study of functional genes; 3. Accurate and efficient information management.

With the development of genome sequencing, the genomes of many species have been sequenced and assembled, so the widespread use of genomic information has greatly facilitated the use of this method. However, at the same time, we have seen that in order to accurately select the genome, the accuracy of the genomic information has room for further improvement. In addition, the extensive discovery of functional genes and in-depth research on their regulatory networks has also greatly facilitated the use of this method. Only when the cause gene locus of the target trait is confirmed and the gene function are clearly defined, can the method be effectively used to improve varieties, so the discovery and functional research of genes need to be further broadened and deepened. At present, in the case of clear gene location, QTL analysis can be used initially to verify the loci affecting traits. Finally, the efficient management of large amounts of genomic information and functional gene information is critical, which guarantees the reliability of information utilization. Therefore, only by satisfying the above points can the method be better used to improve varieties.

We believe that with the further development of genome sequencing, the cost of genome sequencing will be lower and lower, and the accuracy of genome sequencing information will be greatly improved. Then through the exploration of a large number of genes and in-depth study of functions, the use of these information will provide greater convenience for the application of upgrade design breeding in the future. And this method will be more widely used in improving crops, which will be more helpful in solving food problems.

### REFERENCES

Akkaya, M. S., Bhagwat, A. A., and Cregan, P. B. (1992). Length polymorphisms of simple sequence repeat DNA in soybean. Genetics 132, 1131-1139.
Andersen J R, Lubberstedt T (2003) Functional markers in plants. Trends in plant science 8:554-560
A. Sasaki M A, M. Ueguchi-Tanaka H I, A. Nishimura D S, K. Ishiyama, T. Saito M K, G S. Khush, H. Kitano M M (2002) A mutant gibberellin-synthesis gene in rice. NATURE 416:701-702
Ashikari, M., Sakakibara, H., Lin, S. Y., Yamamoto, T., Takashi, T., Nishimura, A., Angeles, E. R., Qian, Q., Kitano, H., and Matsuoka, M. (2005). Cytokinin oxidase regulates rice grain production. Science 309, 741-745.
Bai, X., Huang, Y., Hu, Y., Liu, H., Zhang, B., Smaczniak, C., Hu, G, Han, Z., and Xing, Y. (2017). Duplication of an upstream silencer of FZP increases grain yield in rice. Nat. Plants 3, 885-893.
Bernatzky, R., and Tanksley, S. D. (1986). Toward a saturated linkage map in tomato based on isozymes and random cDNA sequences. Genetics 112, 887-898.
Che, R., Tong, H., Shi, B., Liu, Y., Fang, S., Liu, D., Xiao, Y., Hu, B., Liu, L., Wang, H., et al. (2015). Control of grain size and rice yield by GL2-mediated brassinosteroid responses. Nat. Plants 2, 15195.
Duan, P., Ni, S., Wang, J., Zhang, B., Xu, R., Wang, Y., Chen, H., Zhu, X., and Li, Y. (2015). Regulation of OsGRF4 by OsmiR396 controls grain size and yield in rice. Nat. Plants 2, 15203
Fan C, Xing Y, Mao H, Lu T, Han B, Xu C, Li X, Zhang Q (2006) GS3, a major QTL for grain length and weight and minor QTL for grain width and thickness in rice, encodes a putative transmembrane protein. TAG Theoretical and applied genetics Theoretische and angewandte Genetik 112:1164-1171
Frary, A., Nesbitt, T. C., Grandillo, S., Knaap, E., Cong, B., Liu, J., Meller, J., Elber, R., Alpert, K. B., and Tanksley, S. D. (2000). fw2.2: a quantitative trait locus key to the evolution of tomato fruit size. Science 289, 85-88.
Hospital, F., and Charcosset, A. (1997). Marker-assisted introgression of quantitative trait loci. Genetics 147, 1469-1485.
Hu, J., Wang, Y., Fang, Y., Zeng, L., Xu, J., Yu, H., Shi, Z., Pan, J., Zhang, D., Kang, S., et al. (2015). A rare allele of GS2 enhances grain size and grain yield in rice. Mol. Plant 8, 1455-1465.
Huang R, Jiang L, Zheng J, Wang T, Wang H, Huang Y, Hong Z (2013) Genetic bases of rice grain shape: so many genes, so little known. Trends in plant science 18:218-226
Huang, X., Qian, Q., Liu, Z., Sun, H., He, S., Luo, D., Xia, G, Chu, C., Li, J., and Fu, X. (2009). Natural variation at the DEP1 locus enhances grain yield in rice. Nat. Genet. 41, 494-497.
Huo, X., Wu, S., Zhu, Z., Liu, F., Fu, Y., Cai, H., Sun, X., Gu, P., Xie, D., Tan, L., et al. (2017). NOG1 increases grain production in rice. Nat. Commun. 8, 1497.
Ishimaru, K., Hirotsu, N., Madoka, Y., Murakami, N., Hara, N., Onodera, H., Kashiwagi, T., Ujiie, K., Shimizu, B., Onishi, A., et al. (2013). Loss of function of the IAA-glucose hydrolase gene TGW6 enhances rice grain weight and increases yield. Nat. Genet. 45, 707-711.
James M G, Denyer K, Myers A M (2003) Starch synthesis in the cereal endosperm. Current opinion in plant biology 6:215-222
Jiang, H., Feng, Y., Bao, L., Li, X., Gao, G, Zhang, Q., Xiao, J., Xu, C., and He, Y. (2012a). Improving blast resistance of Jin 23B and its hybrid rice by marker-assisted gene pyramiding. Mol. Breeding 30, 1679-1688.
Jinrong Peng DER, Nigel M. Hartley, George P. Murphy K M D, John E. Flintham, James Beales L J F, Anthony J. Worland, Fatima Pelica D S, Paul Christou, John W. Snape MDGNPH (1999) ‘Green revolution’ genes encode mutant gibberellin response modulators NATURE 400 256-261
Knapp S J (1998) Marker-AssistedSelection as a Strategy for Increasing the Probability of Selecting Superior Genotypes. Crop Sci 38:1164-1174
Li, Z. K., Fu, B. Y., Gao, Y. M., Xu, J. L., Ali, J., Lafitte, H. R., Jiang, Y. Z., Rey, J. D., Vijayakumar, C. H., Maghirang, R., et al. (2005). Genome-wide introgression lines and their use in genetic and molecular dissection of complex phenotypes in rice (Oryza sativa L.). Plant Mol. Biol. 59, 33-52.
Mao H, Sun S, Yao J, Wang C, Yu S, Xu C, Li X, Zhang Q (2010) Linking differential domain functions of the GS3 protein to natural variation of grain size in rice. Proceedings of the National Academy of Sciences of the United States of America 107:19579-19584
Miura K, Ashikari M, Matsuoka M (2011) The role of QTLs in the breeding of high-yielding rice. Trends in plant science 16:319-326
Peleman J D, van der Voort J R (2003) Breeding by Design. Trends in plant science 8:330-334
Qi, P., Lin, Y. S., Song, X. J., Shen, J. B., Huang, W., Shan, J. X., Zhu, M. Z., Jiang, L., Gao, J. P., and Lin, H. X. (2012). The novel quantitative trait locus GL3.1 controls rice grain size and yield by regulating Cyclin-T1-3. Cell Res. 22, 1666-1680.
Ray D K, Ramankutty N, Mueller N D, West P C, Foley J A (2012) Recent patterns of crop yield growth and stagnation. Nature communications 3:1293 Sakamoto T, Matsuoka M (2008) Identifying and exploiting grain yield genes in rice. Current opinion in plant biology 11:209-214
Sasaki, A., Ashikari, M., Ueguchi-Tanaka, M., Itoh, H., Nishimura, A., Swapan, D., Ishiyama, K., Saito, T., Kobayashi, M., Khush, G S., et al. (2002). Green revolution: a mutant gibberellin synthesis gene in rice. Nature 416, 701-702.
She, K. C., Kusano, H., Koizumi, K., Yamakawa, H., Hakata, M., Imamura, T., Fukuda, M., Naito, N., Tsurumaki, Y., Yaeshima, M., et al. (2010). A novel factor FLOURY ENDOSPERM2 is involved in regulation of rice grain size and starch quality. Plant Cell 22, 3280-3294.
Shi-Hua Cheng L-Y C, Jie-Yun Zhuang, Shen-Guang Chen, Xiao-Deng Zhan, Ye-Yang Fan D-FZaS-K M (2007) Super Hybrid Rice Breeding in China: Achievements and Prospects. Journal of Integrative Plant Biology 49 805-810
Song, X. J., Huang, W., Shi, M., Zhu, M. Z., and Lin, H. X. (2007). A QTL for rice grain width and weight encodes a previously unknown RING-type E3 ubiquitin ligase. Nat. Genet. 39, 623-630.
Spielmeyer W, Ellis M H, Chandler P M (2002) Semidwarf (sd-1), “green revolution” rice, contains a defective gibberellin 20-oxidase gene. Proceedings of the National Academy of Sciences of the United States of America 99:9043-9048
Sun, H., Qian, Q., Wu, K., Luo, J., Wang, S., Zhang, C., Ma, Y., Liu, Q., Huang, X., Yuan, Q., et al. (2014). Heterotrimeric G proteins regulate nitrogen-use efficiency in rice. Nat. Genet. 46, 652-656.
Takeda S, Matsuoka M (2008) Genetic approaches to crop improvement: responding to environmental and population changes. Nature reviews Genetics 9:444-457
Tanksley, S. D., and Nelson, J. C. (1996). Advanced backcross QTL analysis: a method for the simultaneous discovery and transfer of valuable QTLs from unadapted germplasm into elite breeding lines. Theor. Appl. Genet. 92, 191-203.
Vos, P., Hogers, R., Bleeker, M., Reijans, M., van de Lee, T., Homes, M., Frijters, A., Pot, J., Peleman, J., Kuiper, M., et al. (1995). AFLP: a new technique for DNA fingerprinting. Nucleic Acids Res. 23, 4407-4414.
Wang H-J C-J (2008) Molecular regulation of sink, source transition in rice leaf sheaths during the heading period. Acta Physiologiae Plantarum 30: 639-649
Wang, E., Wang, J., Zhu, X., Hao, W., Wang, L., Li, Q., Zhang, L., He, W., Lu, B., Lin, H., et al. (2008). Control of rice grain-filling and yield by a gene with a potential signature of domestication. Nat. Genet. 40, 1370-1374.
Wang, H., Ye, S., and Mou, T. (2016). Molecular breeding of rice restorer lines and hybrids for brown planthopper (BPH) resistance using the Bph14 and Bph15 Genes. Rice 9, 53.
Wang, S., Wu, K., Yuan, Q., Liu, X., Liu, Z., Lin, X., Zeng, R., Zhu, H., Dong, G, Qian, Q., et al. (2012). Control of grain size, shape and quality by OsSPL16 in rice. Nat. Genet. 44, 950-954.
Wang Y, Li J (2008) Molecular basis of plant architecture. Annual review of plant biology 59:253-279
Wang, S., Li, S., Liu, Q., Wu, K., Zhang, J., Wang, S., Wang, Y., Chen, X., Zhang, Y, Gao, C., et al. (2015b). The OsSPL16-GW7 regulatory module determines grain shape and simultaneously improves rice yield and grain quality. Nat. Genet. 47, 949-954.
Wang, Y., Xiong, G, Hu, J., Jiang, L., Yu, H., Xu, J., Fang, Y., Zeng, L., Xu, E., Xu, J., et al. (2015a). Copy number variation at the GL7 locus contributes to grain size diversity in rice. Nat. Genet. 47, 944-948.
Wang Y, Li J (2011) Branching in rice. Current opinion in plant biology 14:94-99
Wei, X., Xu, J., Guo, H., Jiang, L., Chen, S., Yu, C., Zhou, Z., Hu, P., Zhai, H., and Wan, J. (2010). DTH8 suppresses flowering in rice, influencing plant height and yield potential simultaneously. Plant Physiol. 153, 1747-1758.
Weng, X., Wang, L., Wang, J., Hu, Y., Du, H., Xu, C., Xing, Y., Li, X., Xiao, J., and Zhang, Q. (2014). Grain number, plant height, and heading date7 is a central regulator of growth, development, and stress response. Plant Physiol. 164, 735-747.
Wei, X., Jiao, G, Lin, H., Sheng, Z., Shao, G, Xie, L., Tang, S., Xu, Q., and Hu, P. (2017).
Williams, J. G, Kubelik, A. R., Livak, K. J., Rafalski, J. A., and Tingey, S. V. (1990). DNA polymorphisms amplified by arbitrary primers are useful as genetic markers. Nucleic acids Res. 18, 6531-6535.
Wittwer C T (2009) High-resolution DNA melting analysis: advancements and limitations. Human mutation 30:857-859
Xing Y, Zhang Q (2010) Genetic and molecular bases of rice yield. Annual review of plant biology 61:421-442
Xue, W., Xing, Y., Weng, X., Zhao, Y., Tang, W., Wang, L., Zhou, H., Yu, S., Xu, C., Li, X., et al. (2008). Natural variation in Ghd7 is an important regulator of heading date and yield potential in rice. Nat. Genet. 40, 761-767.
Yan, W. H., Wang, P., Chen, H. X., Zhou, H. J., Li, Q. P., Wang, C. R., Ding, Z. H., Zhang, Y S., Yu, S. B., Xing, Y. Z., et al. (2011). A major QTL, Ghd8, plays pleiotropic roles in regulating grain productivity, plant height, and heading date in rice. Mol. Plant 4, 319-330.
Zeng, D., Tian, Z., Rao, I, Dong, G, Yang, I, Huang, L., Leng, Y., Xu, J., Sun, C., Zhang, G, et al. (2017). Rational design of high-yield and superior-quality rice. Nat. Plants 3, 17031.
Zuo, J., and Li, J. (2014). Molecular genetic dissection of quantitative trait loci regulating rice grain size. Annu. Rev. Genet. 48, 99-118.
Zhou S R, Yin L L, Xue H W (2013) Functional genomics based understanding of rice endosperm development. Current opinion in plant biology 16:236-246
Zuo J, Li J (2013) Molecular dissection of complex agronomic traits of rice: a team effort by Chinese scientists in recent years. National Science Review 1:253-276

What is claimed is:

1. A method of plant breeding, the method comprising the following steps:
1) Selecting a background variety and a donor variety,
2) Comparing the background variety and the donor variety to identify a module or locus to be improved,
3) Crossing the background variety and the donor variety to obtain a hybrid progeny, backcrossing the hybrid progeny to the background variety to obtain a backcross progeny, and constructing a genetic population using the backcross progeny,
4) Selecting, using molecular markers or a sequencing method, a backcross progeny having chromosomal regions derived from the background variety except for the module or locus to be improved, the molecular markers comprising genome molecular markers and module or locus molecular markers designed according to the selected module or locus,
5) Self-crossing the selected backcross progeny to obtain an improved plant variety.

2. The method of claim 1, wherein step 2) comprises genome sequencing to compare sequences of the module or locus to be improved, such as sequences of an allele, or performing QTL analysis to identity the module or locus to be improved, wherein the module to be improved can be adjusted to a size of, for example, about 50 kb to 5000 kb.

3. The method of claim 1, wherein the molecular markers comprise RFLP, RAPD, SSR, AFLP and SNP; preferably the molecular markers comprise SNP markers; the module or locus molecular markers comprise at least 3 molecular markers, for example 3, 4, 5, 6, 7, 8, 9, 10 or more molecular markers, designed at upstream of the module or locus, within the module or locus, and downstream of the module or locus, respectively.

4. The method of claim 1, wherein the donor variety has an improved trait compared to the background variety, the improved trait comprising for example a yield trait (such as high yield, stable yield and a trait affecting efficiency of light use), a quality trait (such as amino acid composition, sugar composition, protein composition, lipid composition, trace element composition and harmful component composition, such as protease inhibitor, allergen protein and hydrolase composition) and a stress resistance trait (such as disease resistance, antibacterial, antiviral, herbicide resistance, drought resistance, high temperature resistance, cold resistance, insect resistance and a nutrient utilization trait).

5. The method of claim 1, wherein the plant comprises but not limited to Oryza sativa, Zea mays, Triticum aestivum, Phaseolus vulgaris, Glycine max, Brassica spp., Gossypium hirsutum, Helianthus annuus.

6. The method of claim 1, wherein the plant comprises Oryza sativa, the trait comprising a quality trait or a quantitative trait locus (QTL) trait.

7. The method of claim 1, wherein step 3) comprises
Crossing the background variety and the donor variety and continuous backcrossing for 3 or more generations to obtain a BC₃F₁ or later population,
Detecting a genotype of the BC₃F₁ or later population with the genome molecular markers and the module or locus molecular markers, and selecting a BC₃F₁ or later population that has the module or locus derived from the donor variety and has the highest recovery ratio, and
Self-crossing the selected BC₃F₁ or later population to obtain a BC₃F₂ or later population.

8. The method of claim 7, wherein step 3) comprises
Selecting a population with a gene crossover on one side of the module or locus from the BC₃F₂ or later population with the module or locus molecular markers, and self-crossing the selected population to obtain a BC₃F₃ or later population,
Selecting a population with a gene crossover on the other side of the module or locus from the BC₃F₃ or later population,
Eliminating any chromosome fragment derived from the donor variety which is not the module or locus to be improved using backcrossing or self-crossing separation and selecting a population with only an introgressed chromosome fragment of the module or locus to be improved, and self-crossing the selected population to obtain a population with a fixed homozygous module or locus.

9. The method of claim 1, wherein the method comprises repeating steps 1) to 5) one or more times, each time using an improved variety obtained from the previous breeding process as the background variety, and selecting a different module and locus so as to obtain a variety with a plurality of improved modules and loci.

10. A plant variety, obtainable by the method of claim 1, the plant variety being an improved variety compared with the background variety, the improved plant variety comprising an improved module or locus compared with the background variety.
